(19) United States
(12) Patent Application Publication
(10) Pub. No.: US 2019/0192548 A1
(43) Pub. Date: Jun. 27, 2019

(54) USE OF CYCLIC PHOSPHATE SUBSTITUTED NUCLEOSIDE DERIVATIVESFOR THE TREATMENT OF VIRAL DISEASES

(71) Applicants: Stephane BOGEN, Somerset, NJ (US); David DUKHAN, Montpellier (FR); Cyril B. DOUSSON, Montpellier (FR); Christophe Claude PARSY, Jacou (FR)

(72) Inventors: Stephane BOGEN, Somerset, NJ (US); David DUKHAN, Montpellier (FR); Cyril B. DOUSSON, Montpellier (FR); Christophe Claude PARSY, Jacou (FR)

(73) Assignees: MERCK SHARP & DOHME CORP., Rahway, NJ (US); IDENIX PHARMACEUTICALS LLC, Cambridge, MA (US)

(21) Appl. No.: 16/311,410

(22) PCT Filed: Jun. 20, 2017
(86) PCT No.: PCT/US17/38229

§ 371 Date: Dec. 19, 2018

Related U.S. Application Data
(60) Provisional application No. 62/352,087, filed on Jun. 20, 2016.

(51) Int. Cl. A61K 31/7072; A61K 45/06; A61P 31/14

(52) U.S. Cl. CPC A61K 31/7072; A61P 31/14; A61K 45/06

(57) ABSTRACT

The present invention relates to methods of treating or preventing a viral infection using Compounds of Formula (I); or a pharmaceutically acceptable salt thereof, wherein A, B, R¹, R², R³, Q and V are as defined herein. The present invention also relates to compositions comprising a Compound of Formula (I).

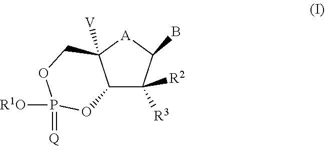

### FIELD OF THE INVENTION

The present invention relates to Compounds of Formula (I), compositions comprising a Compound of Formula (I), and methods of using the Compounds of Formula (I) for treating or preventing viral infection in a patient.

### BACKGROUND OF THE INVENTION

Hepatitis C virus (HCV) infection is a major health problem that leads to chronic liver disease, such as cirrhosis and hepatocellular carcinoma, in a substantial number of infected individuals, estimated to be 2-15% of the world's population. Once infected, about 20% of people clear the virus, but the rest harbor HCV the rest of their lives. Ten to twenty percent of chronically infected individuals eventually develop liver-destroying cirrhosis or cancer. HCV is transmitted parenterally by contaminated blood and blood products, contaminated needles, or sexually and vertically from infected mothers or carrier mothers to their off-spring.

Different approaches to HCV therapy have been taken, which include the inhibition of viral serine proteinase (NS3 protease), helicase, and RNA-dependent RNA polymerase (NS5B), and the development of a vaccine. Current and investigational treatments for HCV infection are reviewed in Poordad et al., Treating hepatitis C: current standard of care. Emerging direct-acting antiviral agents are discussed in Poordad et al., Journal of Viral Hepatitis 19: 449-464 (2012); and Asselah et al., Protease and polymerase inhibitors for the treatment of hepatitis C, Liver International 29(s1): 57-67 (2009). The changing therapeutic landscape for hepatitis C is discussed in Dore, Med. J. Australia 196: 629-632 (2012); and Balsano, Mini Rev. Med. Chem. 8(4): 307-318 (2008). Despite the availability of therapeutic treatment options, chronic HCV infection remains a major healthcare concern. Moreover, there is no established vaccine for HCV. Consequently, there is a need for improved therapeutic agents that effectively combat chronic HCV infection.

The HCV virion is an enveloped positive-strand RNA virus with a single oligoribonucleotide genomic sequence of about 9400 bases which encodes a polyprotein of about 3,000 amino acids. The protein products of the HCV gene consist of the structural proteins C, E1, and E2, and the non-structural proteins NS2, NS3, NS4A, NS4B, NS5A and NS5B. The nonstructural (NS) proteins are believed to provide the catalytic machinery for viral replication.

The NS3 protease releases NS5B, the RNA-dependent RNA polymerase from the polyprotein chain. HCV NS5B polymerase is required for the synthesis of a negative-strand RNA intermediate compound from a positive-strand genomic viral RNA that serves as a template in the replication cycle of HCV. NS5B polymerase is an essential component in the HCV replication complex. See K. Ishi, et al., “Expression of Hepatitis C Virus NS5B Protein: Characterization of Its RNA Polymerase Activity and RNA Binding,” Hepatology, 29:1227-1235 (1999) and V. Lohmann, et al., “Biochemical and Kinetic Analyses of NS5B RNA-Dependent RNA Polymerase of the Hepatitis C Virus,” Virology, 249: 108-118 (1998). Inhibition of HCV NS5B polymerase prevents formation of the double-stranded HCV RNA and therefore constitutes an attractive approach to the development of HCV-specific antiviral therapies.

The development of inhibitors of HCV NS5B polymerase with potential for the treatment of HCV infection has been reviewed in Poordad et al. (2012), supra; Asselah et al. (2009), supra; and Chatel-Chaix et al. Direct-acting and host-targeting HCV inhibitors: current and future directions. Current Opinion in Virology, 2:588-598 (2012). The activity of purine ribonucleosides against HCV polymerase was reported by A.E. Eldrup et al., “Structure-Activity Relationship of Purine Ribonucleosides for Inhibition of HCV RNA-Dependent RNA Polymerase,” J. Med. Chem., 47:2283-2295 (2004).

### SUMMARY OF THE INVENTION

In one aspect, the present invention provides a method for treating a patient infected with HCV, said method comprising administering a compound of formula (I), or a pharmaceutically acceptable salt thereof, in an amount effective to treat infection by HCV in said patient, wherein formula (I) is:

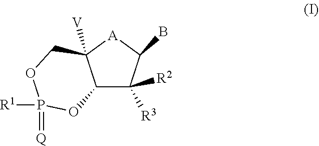

wherein:

A is O or S;

B is selected from:

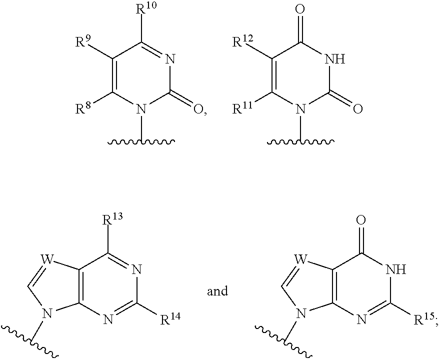

Q is O or S;

V is hydrogen, halogen or amino;

W is N, CH or CF;

R¹ is C₁-C₆ alkoxy, —O—(C₁-C₆ alkylene)-S—C(O)—(C₁-C₆ alkyl),

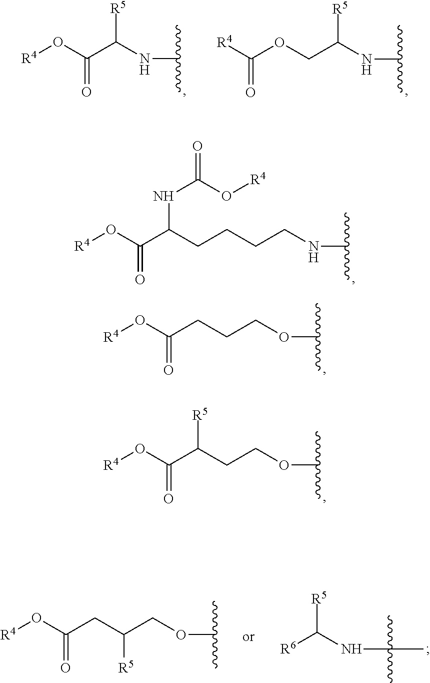

R² is halo;

R³ is halo;

each occurrence of R⁴ is independently selected from C₁-C₁₀ alkyl, C₃-C₁₀ cycloalkyl, aryl or —(C₁-C₆ alkylene)-aryl;

R⁵ is C₁-C₁₀ alkyl or —COOR⁷;

R⁶ is selected from —(C₁-C₁₀ alkylene)-C(O)O—(C₁-C₁₀ alkyl), —OC(O)—(C₃-C₁₀ cycloalkyl), aryl, aryloxy, —(C₁-C₁₀ alkylene)-aryl, 5 or 6-membered monocyclic heteroaryl , 9 or 10-membered bicyclic heteroaryl, —(C₁-C₁₀ alkylene)-(5 or 6-membered monocyclic heteroaryl) and —(C₁-C₁₀ alkylene)-(9 or 10-membered bicyclic heteroaryl);

R⁷ is C₁-C₁₀ alkyl, C₃-C₁₀ cycloalkyl, aryl or —(C₁-C₆ alkylene)-aryl;

R⁸, R⁹, R¹¹ and R¹² are each independently selected from H, C₁-C₆ alkyl, C₁-C₆ haloalkyl, C₁-C₆ hydroxyalkyl, halo, —OR¹⁶, —SR¹⁶ and —N(R¹⁶)₂;

R¹⁰, R¹³, R¹⁴ and R¹⁵ are each independently selected from H, C₁-C₆ alkyl, C₂-C₆ alkenyl, C₂-C₆ alkynyl, C₃-C₇ cycloalkyl, 5- or 6-membered monocyclic heteroaryl, 9- or 10-membered bicyclic heteroaryl, halo, —OR¹⁶, —SR¹⁶, —S(O)R¹⁶, —S(O)₂R¹⁶, —S(O)₂N(R¹⁶)₂, —NHC(O)OR¹⁶, —NHC(O)N(R¹⁶)₂, C₁-C₆ haloalkyl, C₁-C₆ hydroxyalkyl, —O—(C₁-C₆ haloalkyl), —CN, —NO₂, —N(R¹⁶)₂, —NH(C₁-C₆ alkylene)-(5- or 6-membered monocyclic heteroaryl), —NH(C₁-C₆ alkylene)-(9- or 10-membered bicyclic heteroaryl), —C(O)R¹⁶, —C(O)OR¹⁶, —C(O)N(R¹⁶)₂ and —NHC(O)R¹⁶, wherein said C₂-C₆ alkenyl group and said C₂-C₆ alkynyl group may be optionally substituted with halo;

each occurrence of R¹⁶ is independently selected from H, C₁-C₆ alkyl, C₁-C₆ haloalkyl, C₁-C₆ hydroxyalkyl, —(C₁-C₃ alkylene)ₘ—(C₃-C₇ cycloalkyl), —(C₁-C₃ alkylene)ₘ—(C₆-C₁₀ aryl), —(C₁-C₃ alkylene)ₘ—(4 to 7-membered heterocycloalkyl), —(C₁-C₃ alkylene)ₘ—(5- or 6-membered monocyclic heteroaryl) and —(C₁-C₃ alkylene)ₘ-(9- or 10-membered bicyclic heteroaryl); and

each occurrence of m is independently 0 or 1.

The Compounds of Formula (I) or pharmaceutically acceptable salts thereof may be useful, for example, for inhibiting HCV viral replication or replicon activity, for inhibiting HCV NS5B activity, and for treating or preventing HCV infection in a patient. Without being bound by any specific theory, it is believed that the Compounds of Formula(I) inhibit HCV viral replication by inhibiting HCV NS5B.

Accordingly, the present invention provides methods for treating or preventing HCV infection in a patient, comprising administering to the patient an effective amount of at least one Compound of Formula(I).

The details of the invention are set forth in the accompanying detailed description set forth below.

Other embodiments, aspects and features of the present invention are either further described in or will be apparent from the ensuing description, examples and appended claims.

### DETAILED DESCRIPTION OF THE INVENTION

The present invention relates to Compounds of Formula(I), compositions comprising a Compound of Formula(I), and methods of using the Compounds of Formula(I) for treating or preventing HCV infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc.

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Compound of Formula(I) and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood or severity of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 10 carbon atoms (C₁-C₁₀ alkyl), from about 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted with one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “C₁-C₆ alkoxy” as used herein, refers to a group having the formula —O—(C₁-C₆ alkyl), where the term “C₁-C₆ alkyl” is defined above herein.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted with one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted with one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—,—CH(CH₃)CH₂CH₂—, —CH(CH₃)— and —CH₂CH(CH₃)CH₂—. In one embodiment, an alkylene group has from 1 to about 6 carbon atoms (C₁-C₆ alkylene). In another embodiment, an alkylene group has from 1 to about 10 carbon atoms (C₁-C₁₀ alkylene). In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. In one embodiment, an aryl group can be optionally fused to a cycloalkyl or cycloalkanoyl group. Non-limiting examples of aryl groups include phenyl and naphthyl. In one embodiment, an aryl group is phenyl. The term “aryloxy” as used herein, refers to a group having the formula —O-aryl, where the term “aryl” is defined above herein.

The term “cycloalkyl,” as used herein, refers to a non-aromatic mono- or multicyclic ring system comprising from 3 to about 10 ring carbon atoms (C₃-C₁₀ cycloalkyl). In one embodiment, a cycloalkyl contains from about 5 to about 10 ring carbon atoms (C₅-C₁₀ cycloalkyl). In another embodiment, a cycloalkyl contains from 3 to about 7 ring atoms (C₃-C₇ cycloalkyl). In another embodiment, a cycloalkyl contains from about 5 to about 6 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantly. In one embodiment, a cycloalkyl group is unsubstituted. The term “3 to 6-membered cycloalkyl” refers to a cycloalkyl group having from 3 to 6 ring carbon atoms. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

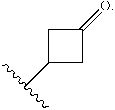 [SMILES: *C1CC(=O)C1]

The term “halo,” as used herein, means —F, —Cl, —Br or —I.

The term “haloalkyl,” as used herein, refers to an alkyl group as defined above, wherein one or more of the alkyl group's hydrogen atoms have been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “5 or 6-membered monocyclic heteroaryl,” as used herein, refers to an aromatic monocyclic ring system comprising about 5 to about 6 ring atoms, wherein from 1 to 4 of the ring atoms is independently O, N or S and the remaining ring atoms are carbon atoms. A 5 or 6-membered monocyclic heteroaryl group is joined via a ring carbon atom, and any nitrogen atom of a heteroaryl can be optionally oxidized to the corresponding N-oxide. The term “5 or 6-membered monocyclic heteroaryl” also encompasses a 5 or 6-membered monocyclic heteroaryl group, as defined above, which is fused to a benzene ring. Non-limiting examples of 5 or 6-membered monocyclic heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, benzofurazanyl, indolyl, azaindolyl, benzimidazolyl, benzothienyl, imidazolyl, benzimidazolyl, thienopyridyl, thienopyrimidyl, pyrrolopyridyl, imidazopyridyl, isoquinolinyl, 1,2,4-triazinyl, benzothiazolyl and the like, and all isomeric forms thereof. The term “9 or 10-membered bicyclic heteroaryl,” as used herein, refers to an aromatic bicyclic ring system comprising about 9 to about 10 ring atoms, wherein from 1 to 4 of the ring atoms is independently O, N or S and the remaining ring atoms are carbon atoms. A 9 or 10-membered bicyclic heteroaryl group is joined via a ring carbon atom, and any nitrogen atom of a heteroaryl can be optionally oxidized to the corresponding N-oxide. Non-limiting examples of 9 or 10-membered bicyclic heteroaryls include imidazo[1,2-a]pyridinyl, imidazo[2,1-b]thiazolyl, benzofurazanyl, indolyl, azaindolyl, benzimidazolyl, benzothienyl, quinolinyl, benzimidazolyl, quinazolinyl, pyrrolopyridyl, imidazopyridyl, isoquinolinyl, benzoazaindolyl, benzothiazolyl, and the like, and all isomeric forms thereof.

The term “heterocycloalkyl,” as used herein, refers to a non-aromatic monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

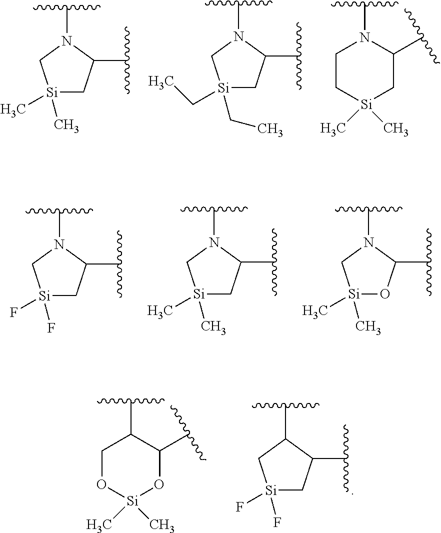

A ring carbon atom of a heterocycloalkyl group may be functionalized as a carbonyl group. An illustrative example of such a heterocycloalkyl group is:

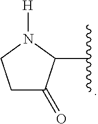 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic cycloalkyl” refers to a monocyclic heterocycloalkyl group having from 3 to 6 ring atoms. The term “4 to 6-membered monocyclic cycloalkyl” refers to a monocyclic heterocycloalkyl group having from 4 to 6 ring atoms. The term “7 to 11-membered bicyclic heterocycloalkyl” refers to a bicyclic heterocycloalkyl group having from 7 to 11 ring atoms. Unless otherwise indicated, an heterocycloalkyl group is unsubstituted.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, N.Y.

When any substituent or variable (e.g., alkyl, R⁶, le, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results directly from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Compound of Formula(I) or a pharmaceutically acceptable salt of the compound. The transformation may occur by various mechanisms (e.g., by metabolic or chemical processes), such as, for example, through hydrolysis in blood.

For example, if a Compound of Formula(I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Compound of Formula(I) contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-a-aminoacyl, where each α-aminoacyl group is independently selected from the naturally occurring L-amino acids, or glycosyl (the radical resulting from the removal of a hydroxyl group of the hemiacetal form of a carbohydrate). Other non-limiting example of alcohol-derived prodrugs include —P(O)(OH)₂; —P(O)(—O—C₁-C₆alkyl)₂; —P(O)(—NH—(α-aminoacyl group))(—O-aryl); —P(O)(—O—(C₁-C₆ alkylene)—S-acyl)(-NH-arylalkyl);and those described in U.S. Pat. No. 7,879,815; International Publication Nos. WO2005/003047, WO2008/082602, WO2010/0081628, WO2010/075517 and WO2010/075549; Mehellou, Chem. Med. Chem., 5:1841-1842 (2005); Bobeck et al., Antiviral Therapy 15:935-950 (2010); Furman et al., Future Medicinal Chemistry, 1:1429-1452 (2009); and Erion, Microsomes and Drug Oxidations, Proceedings of the International Symposium, 17th, Saratoga Springs, N.Y., United States, July 6-10, 2008, 7-12 (2008).

If a Compound of Formula(I) incorporates an amine functional group, a prodrug can be formed by the replacement of a hydrogen atom in the amine group with a group such as, for example, R-carbonyl-, RO-carbonyl-, NRR'-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N-(C₁-C₆)alkylaminoalkyl; —C(Y⁴)Y⁵ wherein Y⁴ is H or methyl and Y⁵ is mono-N- or di-N,N-(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours. , 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Compounds of Formula(I) can form salts which are also within the scope of this invention. The term “salt(s)”, as employed herein, denotes acidic salts formed with inorganic and/or organic acids, as well as basic salts formed with inorganic and/or organic bases. In addition, when a Compound of Formula(I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Compound of Formula(I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, N.Y.; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures may be separated into their individual diastereomers on the basis of their physical chemical differences by methods well-known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Compounds of Formula(I) may be atropisomers (e.g., substituted biaryls) and are considered as part of this invention. Enantiomers can also be directly separated using chiral chromatographic techniques.

It is also possible that the Compounds of Formula(I) may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Compound of Formula(I) incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) may be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Compounds of Formula(I), and of the salts, solvates, hydrates, esters and prodrugs of the Compounds of Formula(I), are intended to be included in the present invention.

Formula I includes a chiral amino acid residue linked to a 5′-phosphoramidate group. Those of skill in the art will recognize that the amino acid residue has R stereochemistry at the carbon bonded to R¹⁰; i.e., that it is a D-amino acid residue.

Some compounds provided herein are based, at least in part, on the discovery that D-amino acid phosphoramidate prodrugs can provide superior human pharmacokinetics including superior accumulation of active nucleoside and nucleotide analogs in target cells, such as liver cells. In certain embodiments, the compounds provided herein are D-amino acid, Rp phosphoramidate compounds. In certain embodiments, the compounds provided herein are D-amino acid, Sₚ phosphoramidate compounds. Any compound provided herein is preferably in the form of a composition that is substantially free of other stereoisomers of the compound, as described herein.

The following abbreviations are used herein:
Ac Acetyl
aq Aqueous
Boc or BOC tert-butoxycarbonyl
Bu Butyl
t-Bu tertiary butyl
t-BuMgCl t-butyl magnesium chloride
calc'd Calculated
Celite/celite diatomaceous earth
DBU 1,8-diazabicyclo(5.4.0)undec-7-ene
DCE 1,2-dichloroethane
DCM Dichloromethane
DIEA or DIPEA N,N-diisopropylethylamine
DMA 1,2-dimethylacetamide
DMAP 4-dimethylaminopyridine
DMF Dimethylformamide
DMSO dimethyl sulfoxide
dMTr 4,4′-dimethoxytrityl
dMTrCl 4,4′-dimethoxytrityl chloride
EDCI 1-ethyl-3-(3-dimethylaminopropyl)carbodiimide
EDTA ethylenediamine tetraacetic acid
EGTA ethylene glycol-bis(β-aminoethyl ether)-N,N,N′,N′-tetraacetic acid
ESI electrospray ionization
Et Ethyl
Et₂O diethyl ether
EtOH Ethanol
EtOAc ethyl acetate
Et₃N Trimethylamine
Et₃ SiH Triethylsilane
GAPDH glyceraldehyde-3-phosphate dehydrogenase
HPLC high-performance liquid chromatography
IPA Isopropanol
iPr Isopropyl
iPrOAc isopropyl acetate
LC liquid chromatography
LC/MS liquid chromatography mass spectrometry
Me Methyl
MeOH Methanol
MS mass spectrometry
NMI 1-methylimidazole
NMR nuclear magnetic resonance spectroscopy
Pd/C palladium on carbon
Ph Phenyl
Pr Propyl
RT room temperature
Rt retention time
RT-PCR Real-time polymerase chain reaction
sat Saturated
TMS Trimethyl silyl
TMSCl trimethylsilyl chloride
t-Bu tent-butyl
TFA trifluoroacetic acid
TFAA trifluoroacetic anhydride
THF Tetrahydrofuran
TLC thin layer chromatography
TMS Trimethylsilyl

### Use of The Compounds of Formula (I)

The present invention provides methods of using the Compounds of Formula(I) to treat HCV infection in a patient, wherein Formula(I) is:

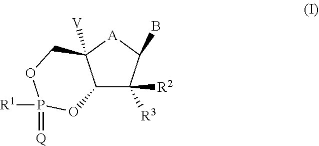

or a pharmaceutically acceptable salt thereof, wherein A, B, R¹, R², R³, Q and V are defined above for the Compounds of Formula (I).

In one embodiment, A is O.

In another embodiment, A is S.

In one embodiment, B is selected from guanine, cytosine, adenine and uracil.

In another embodiment, B is:

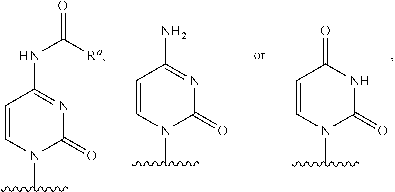

wherein le is C₁-C₆ alkyl or C₆-C₁₀ aryl.

In another embodiment, B is:

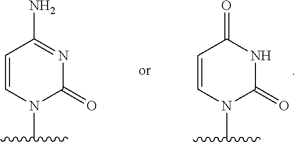 [SMILES: *n1ccc(N)nc1=O ; *n1ccc(=O)[nH]c1=O]

In still another embodiment, B is:

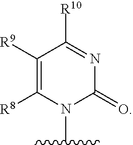

In another embodiment, B is:

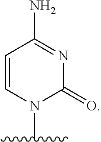 [SMILES: *n1ccc(N)nc1=O]

In another embodiment, B is:

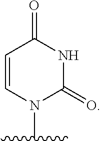 [SMILES: *n1ccc(=O)[nH]c1=O]

In yet another embodiment, B is

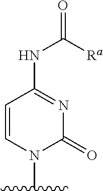

and R^{a} is C₁-C₆ alkyl.

In another embodiment, B is

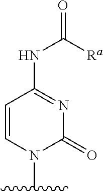

and R^{a} is C₆-C₁₀ aryl.

In one embodiment, Q is O.

In another embodiment, Q is S.

In one embodiment, V is H.

In another embodiment, V is F.

In one embodiment, R¹ is C₁-C₆ alkoxy, —O—(C₁-C₆ alkylene)-S—C(O)—(C₁-C₆ alkyl),

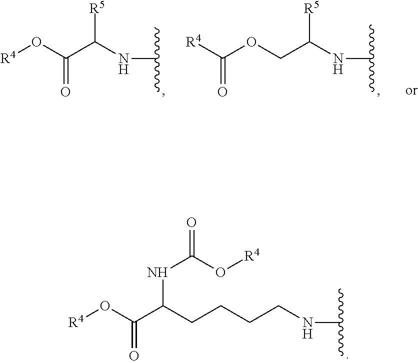

In one embodiment, R¹ is C₁-C₆ alkoxy, —O—(C₁-C₆ alkylene)-S—C(O)—(C₁-C₆ alkyl),

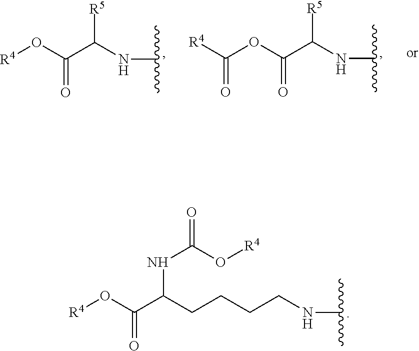

In one embodiment, R¹ is:

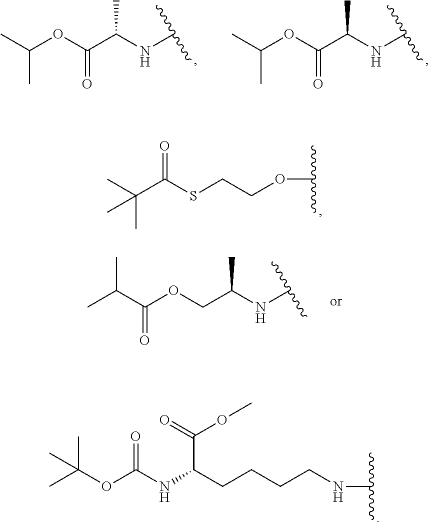

In one embodiment, R¹ is C₁-C₆ alkoxy or

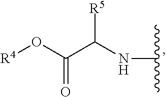

wherein each of R⁴ and R⁵ is independently C₁-C₆ alkyl.

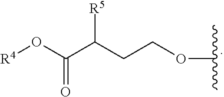

In one embodiment, R¹ is

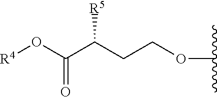

In one embodiment, R¹ is

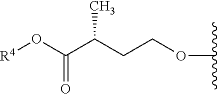

In one embodiment, R¹ is

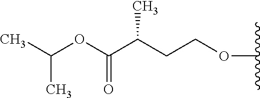 [SMILES: *OCC[C@@H](C)C(=O)OC(C)C]

In one embodiment, R¹ is

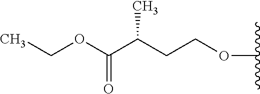 [SMILES: *OCC[C@@H](C)C(=O)OCC]

In one embodiment, R¹ is

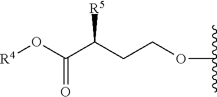

In one embodiment, R¹ is

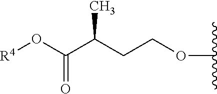

In one embodiment, R¹ is

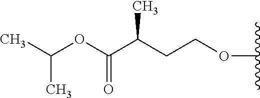 [SMILES: *OCC[C@H](C)C(=O)OC(C)C]

In one embodiment, R¹ is

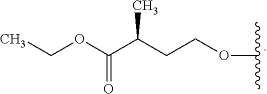 [SMILES: *OCC[C@H](C)C(=O)OCC]

In one embodiment, R¹ is

In one embodiment, R² is F.

In another embodiment, R² is Cl.

In one embodiment, R³ is F.

In another embodiment, R³ is Cl.

In one embodiment, each of R² and R³ is Cl.

In another embodiment, each of R² and R³ is F.

In one embodiment, R⁴ is C₁-C₁₀ alkyl.

In one embodiment, R⁴ is C₁-C₆ alkyl.

In one embodiment, R⁴ is methyl, ethyl, propyl or isopropyl.

In another embodiment, R⁴ is isopropyl.

In another embodiment, R⁴ is ethyl.

In one embodiment, R⁵ is C₁-C₆ alkyl.

In one embodiment, R⁵ is methyl, ethyl, propyl or isopropyl.

In another embodiment, R⁵ is isopropyl.

In one embodiment, R⁵ is methyl.

In one embodiment, R⁵ is (S)-isopropyl.

In one embodiment, R⁵ is (R)-isopropyl.

In one embodiment, R⁵ is (S)-methyl.

In one embodiment, R⁵ is (R)-methyl.

In one embodiment, V is H and each of R² and R³ is F.

In another embodiment, V is F and each of R² and R³ is F.

In one embodiment, A is O, V is hydrogen and each of R² and R³ is F.

In one embodiment:

Q is O;

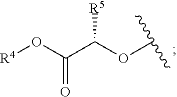

R¹ is —(C₁-C₆ alkylene)-aryl or

R⁴ is C₁-C₁₀ alkyl; and

R⁵ is C₁₋₆alkyl.

In another embodiment:

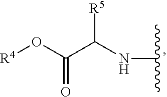

R¹ is C₁-₆ alkoxy or

wherein each of R⁴ and R⁵ is independently C₁-C₆ alkyl; and

Q is O.

In another embodiment:

R¹ is C₁₋₆ alkoxy or

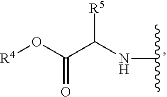

wherein each of R⁴ and R⁵ is independently C₁-C₆ alkyl; and

Q is O;

A is O;

V is hydrogen;

each of R² and R³ is F;

B is

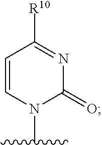

and R¹⁰ is —NHC(O)—(C₁-C₆ alkyl), —NH₂ or —OH.

In still another embodiment :

R¹ is isopropoxy;

Q is O;

A is O;

V is hydrogen;

each of R² and R³ is F;

B is

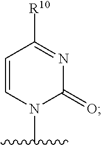

and R¹⁰ is —NHC(O)—(C₁-C₆ alkyl), —NH₂ or —OH.

In one embodiment:

R¹ is

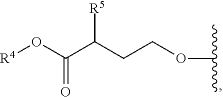

wherein each of R⁴ and R⁵ is independently C₁-C₆ alkyl; and

Q is O.

In another embodiment:

R¹ is

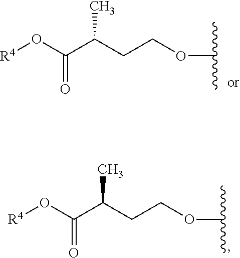

wherein R⁴ is ethyl or isopropyl; and

Q is O;

A is O;

V is hydrogen; each of R² and R³ is F;

B is

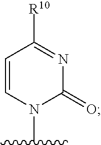

and

R¹⁰ is —NHC(O)—(C₁-C₆ alkyl), —NHC(O)—(C₆-C₁₀ aryl), —NH₂ or —OH.

In one embodiment, the compound of formula (I) used in the methods of the present invention is selected from:

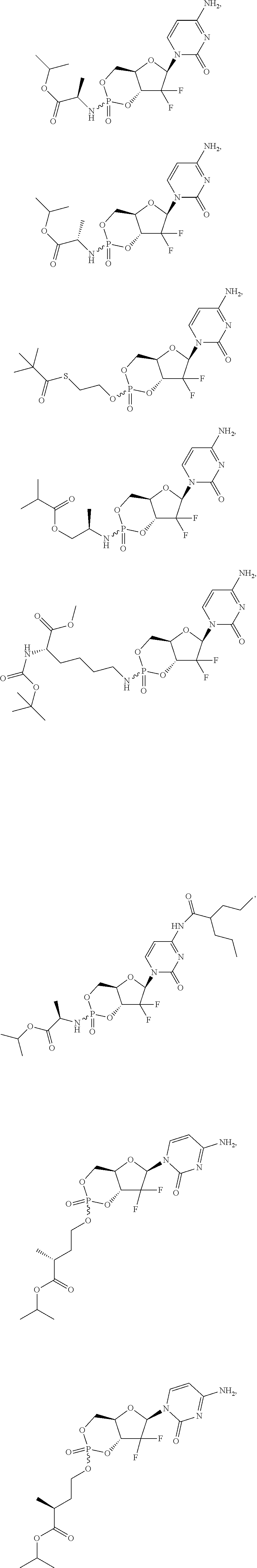

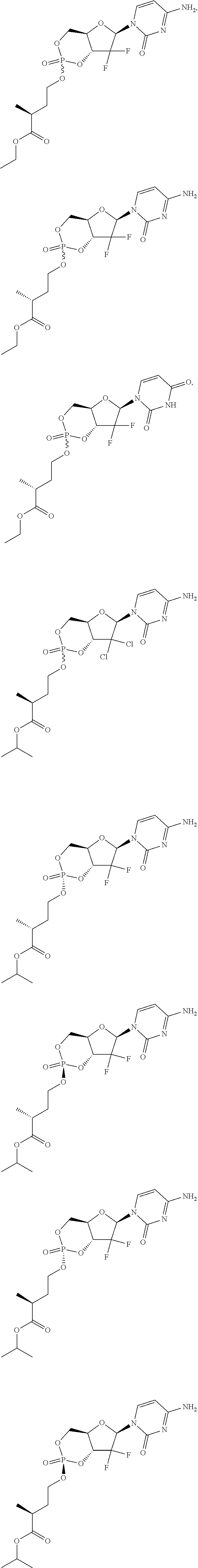

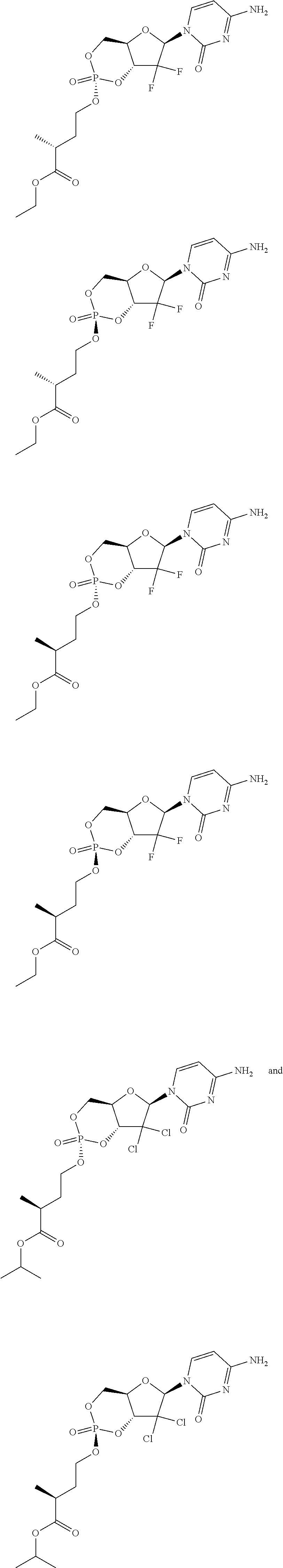

or a pharmaceutically acceptable salt thereof.

In one embodiment, variables A, B, R², R³, Q and V for the Compounds of Formula (I) are selected independently of each other.

In another embodiment, the Compounds of Formula (I) are in substantially purified form.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Other embodiments of the present invention include the following:

(a) A pharmaceutical composition comprising an effective amount of a Compound of Formula (I), or a pharmaceutically acceptable salt thereof, and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of HCV protease inhibitors, HCV NS5B polymerase inhibitors and HCV NS5A inhibitors.

(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.

(e) The combination of (d), wherein the HCV antiviral agent is an antiviral selected from the group consisting of HCV protease inhibitors, HCV NS5B polymerase inhibitors and HCV NS5A inhibitors.

(f) A method of inhibiting HCV replication in a subject in need thereof which comprises administering to the subject an effective amount of a Compound of Formula (I).

(g) A method of treating HCV infection and/or reducing the likelihood or severity of symptoms of HCV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HCV antiviral agents, immunomodulators, and anti-infective agents.

(i) The method of (h), wherein the HCV antiviral agent is an antiviral selected from the group consisting of HCV protease inhibitors, HCV NS5B polymerase inhibitors and HCV NS5A inhibitors.

(j) A method of inhibiting HCV replication in a subject in need thereof which comprises administering to the subject the pharmaceutical composition of (a), (b) or (c) or the combination of (d) or (e).

(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

Additional embodiments of the invention include the pharmaceutical compositions, combinations and methods set forth in (a)-(k) above and the uses set forth in the discussion below, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Scheme A below. Alternative synthetic pathways and analogous structures will be apparent to those skilled in the art of organic synthesis.

Scheme A shows a method useful for making nucleoside compounds of formula (iii), which correspond to the Compounds of Formula (I).

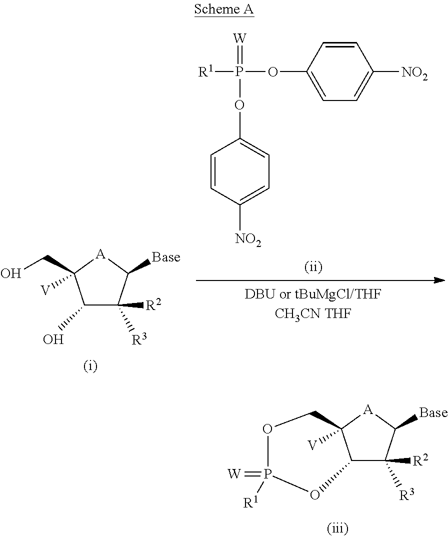

A nucleoside compound of formula (i) can be reacted with a compound of formula (ii) in the presence of DBU or t-BuMgCl to provide the compounds of formula (iii), which correspond to the compounds of formula (I).

Scheme B shows an alternative method useful for making nucleoside compounds of formula (iii), which correspond to the Compounds of Formula (I).

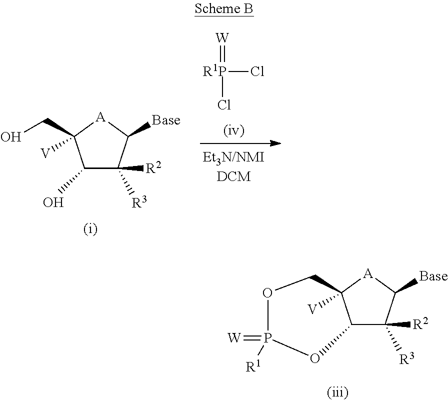

A nucleoside compound of formula (i) can be reacted with a compound of formula (iv) in the presence of trimethylamine and NMI to provide the compounds of formula (iii), which correspond to the compounds of formula (I).

### EXAMPLES

### General Procedures

Reactions sensitive to moisture or air were performed under nitrogen or argon atmosphere using anhydrous solvents and reagents. The progress of reactions was determined using either analytical thin layer chromatography (TLC) usually performed with E. Merck pre-coated TLC plates, silica gel 60E-254, layer thickness 0.25 mm or liquid chromatography-mass spectrometry (LC-MS).

The analytical UPLC-MS system used consisted of a Waters SQD2 platform with electrospray ionization in positive and negative detection mode with an Acquity UPLC I-class solvent manager, column manager, sample manager and PDA detector. The column used for standard methods was a CORTECS UPLC C18 1.6 μm, 2.1×30 mm, and the column used for polar methods was an ACQUITY UPLC HSST3 1.8 μm, 2.1×30 mm, the column temperature was 40° C., the flow rate was 0.7 mL/min, and injection volume was 1 μL. UV detection was in the range 210-400 nm. The mobile phase consisted of solvent A (water plus 0.05% formic acid) and solvent B (acetonitrile plus 0.05% formic acid) with different gradients for 4 different methods: 1/Starting with 99% solvent A for 0.2 minutes changing to 98% solvent B over 1 minutes, maintained for 0.4 minutes, then reverting to 99% solvent A over 0.1 min; 2/Starting with 99% solvent A for 0.5 minutes changing to 98% solvent B over 3.7 minutes, maintained for 0.4 minutes, then reverting to 99% solvent A over 0.1 min; 3/Starting with 100% solvent A for 0.4 minutes changing to 98% solvent B over 0.9 minutes, maintained for 0.3 minutes, then reverting to 100% solvent A over 0.1 min; 4/Starting with 100% solvent A for 0.8 minutes changing to 98% solvent B over 3.4 minutes, maintained for 0.4 minutes, then reverting to 100% solvent A over 0.1 minutes.

The analytical LC-MS system used consisted of a Agilent 6140 quadrupole LC/MS platform with electrospray ionization in positive and negative detection mode with an Agilent 1200 Series solvent manager, column manager, sample manager and PDA detector. The column for standard method was Purospher® STAR RP-18 endcapped 2 μm, Hibar® HR 50-2.1, the column temperature was 60° C., the flow rate was 0.8 mL/min, and injection volume was 0.5-5 μL. UV detection was in the range 210-400 nm. The mobile phase consisted of solvent A (water plus 0.05% formic acid) and solvent B (acetonitrile plus 0.05% formic acid) with different gradients for 2 different methods: 1) Starting with 98% solvent A changing to 100% solvent B over 1.8 minutes, maintained for 0.8 min; 2) Starting with 98% solvent A changing to 100% solvent B over 5.8 minutes, maintained for 0.3 minutes.

Preparative HPLC purifications were usually performed using a mass spectrometry directed system. Usually they were performed on a Waters Chromatography Workstation (MassLynx V4.1) configured with LC-MS System Consisting of: Waters ZQ™ 2000 (quad MS system with Electrospray Ionization), Waters 2545 Gradient Pump, Waters 2767 Injecto/Collector, Waters 2998 PDA Detector, the MS Conditions of: 100-1400 amu, Positive Electrospray, Collection Triggered by MS, and a Waters SUNFIRE® C-18 5 micron, 19 mm (id)×150 mm column. The mobile phases consisted of mixtures of acetonitrile (5-95%) in water containing 0.02% formic acid. Flow rates were maintained at 20 mL/min, the injection volume was 500 to 3000 μL, and the UV detection range was 210-400 nm. Mobile phase gradients were optimized for the individual compounds. Preparative HPLC were also performed on a Gilson system GX-281 (Trilution). The column was a Waters SUNFIRE® Prep C18 5 μm OBD, dimension 50×150 mm. The mobile phase consisted of acetonitrile (5-50%) in water containing 0.02% HCOOH over 60 minutes. Flow rates were maintained at 117 mL/min, the injection volume was 1000 to 7000 μL, and the UV detection range was 260 nm.

Reactions performed using microwave irradiation were normally carried out using an Emrys Optimizer manufactured by Personal Chemistry, or an Initiator manufactured by Biotage. Concentration of solutions was carried out on a rotary evaporator in vacuo. Flash chromatography was usually performed using a Biotage® Flash Chromatography apparatus

(Isolera) on silica gel (15-45 μ, 40-63 μu, or spheric silica) in pre-packed cartridges of the size noted. ¹H NMR spectra were acquired at 400 MHz or 500 MHz spectrometers in CDCl₃ solutions unless otherwise noted. Chemical shifts were reported in parts per million (ppm). Tetramethylsilane (TMS) was used as internal reference in CDCl₃ solutions, and residual CH₃OH peak or TMS was used as internal reference in CD₃OD solutions. Coupling constants (J) were reported in hertz (Hz). Chiral analytical chromatography was performed on one of CHIRALPAK® AS, CHIRALPAK®AD, CHIRALCEL® OD, CHIRALCEL® IA, or CHIRALCEL® OJ columns (250×4.6 mm) (Daicel Chemical Industries, Ltd.) with noted percentage of either ethanol in hexane (%EtOH/Hex) or isopropanol in heptane (% IPA/Hep) as isocratic solvent systems. Chiral preparative chromatography was conducted on one of of CHIRALPAK AS, of CHIRALPAK AD, CHIRALCEL® OD, CHIRALCE® IA, CHIRALCEL® OJ columns (20×250 mm) (Daicel Chemical Industries, Ltd.) with desired isocratic solvent systems identified on chiral analytical chromatography or by supercritical fluid (SFC) conditions.

### Example 1

General Methods Useful for the Preparation of Compounds 1-5

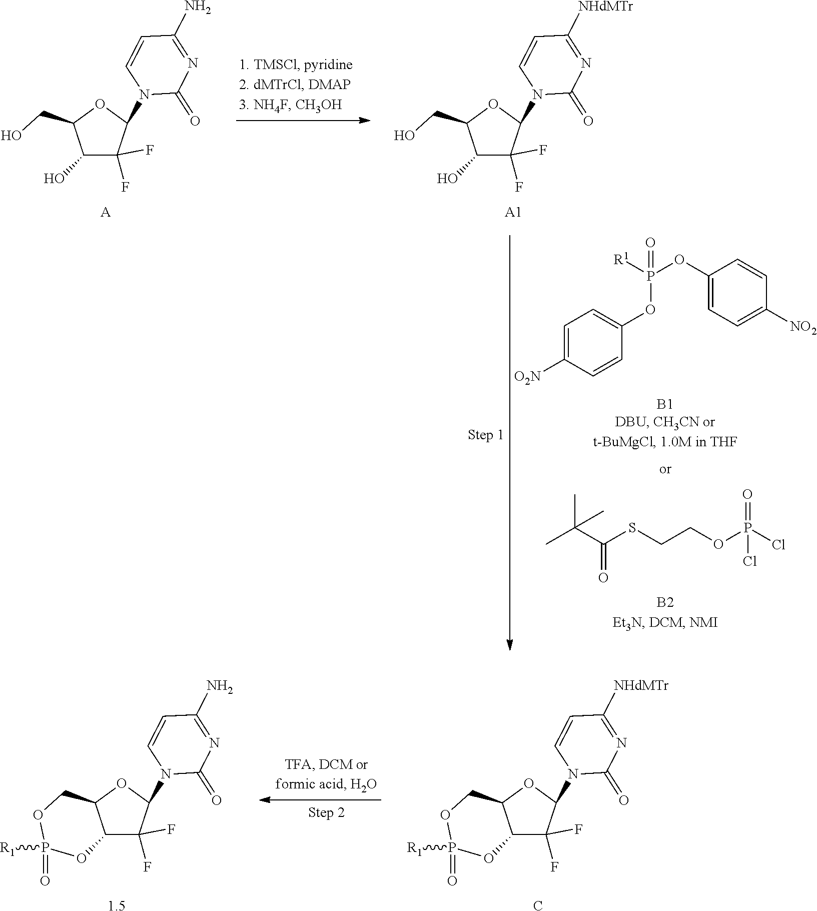

TABLE 1

| Com- | | General |
|---|---|---|
| pound | | Method |
| No. | R¹ | Used |
| 1 | 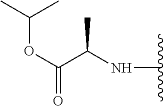 [SMILES: *N[C@H](C)C(=O)OC(C)C] | A or B |
| 2 | 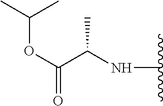 [SMILES: *N[C@@H](C)C(=O)OC(C)C] | A or B |
| 3 | 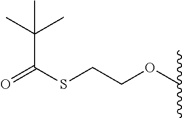 [SMILES: *OCCSC(=O)C(C)(C)C] | C |
| 4 | 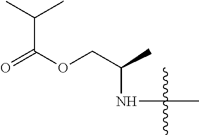 [SMILES: *N[C@H](C)COC(=O)C(C)C] | A or B |
| 5 | 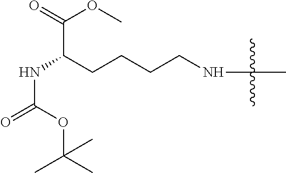 [SMILES: *NCCCC[C@H](NC(=O)OC(C)(C)C)C(=O)OC] | B |

### Method for Preparation of Compound A1

Diol starting material A (50.00 g, 0.19 mol) and pyridine (900 mL) were stirred for 1 h at room temperature, and then, cooled to −5° C. Trimethylsilyl chloride (145 mL, 1.14 mol) was added dropwise over a period of 20 min. The reaction mixture was allowed to stir for 1 h at −5° C., and then, warmed to room temperature. The reaction mixture was allowed to stir for 30 min at room temperature. 4-(Dimethylamino)pyridine (23.20 g, 0.19 mol) and monomethoxytrityl chloride (73.40 g, 0,24 mol) were added to the reaction mixture and the resulting reaction mixture was warmed to 57° C. overnight. Most of the solvent was removed in vacuo and the resulting residue obtained was azeotroped with toluene (500 mL). The residue obtained was dissolved in DCM (700 mL) and a solution of saturated sodium bicarbonate (900 mL) was added dropwise to it while stirring. The reaction mixture was allowed to stir for 30 min. The aqueous layer was further extracted with DCM (450 mL). The combined organic layers were dried over sodium sulfate, filtered and concentrated in vacuo. The residue obtained was dissolved in methanol (700 mL) and ammonium fluoride was added. The reaction mixture was allowed to stir at 60° C. for 4 h. Water (700 mL) was added to the reaction mixture. The mixture was heated at 50° C. for 10 min., then was cooled to room temperature. Solvent was partially removed in vacuo. The residue obtained was azeotroped with methanol and toluene. The crude product was purified using silica gel flash column chromatography (DCM/methanol 6%) to provide compound A1.

### (General Method A)

### Step 1 - Preparation of Intermediate Compound C

To a solution of compound Al in THF (25 mL/mmol of Al) at 0° C. was added a solution of tert-butylmagnesium chloride (1M in THF, 3 eq.) followed by the appropriate reagent Bl (1.2 eq.) in DCM. The reaction mixture was allowed to stir at room temperature overnight. The reaction mixture was diluted with ethyl acetate and quenched with brine. The aqueous layer was extracted with ethyl acetate. The combined organic layers were dried, filtered and concentrated in vacuo. The crude residue obtained was purified using flash chromatography on silica gel (DCM/methanol: 0 to 20%) to provide the appropriate intermediate of formula C.

### Step 2

To a solution of C (1.0 eq) in DCM (30 mL/mmol of C) was added trifluoroacetic acid (12 eq.) under nitrogen. The reaction mixture was allowed to stir at room temperature between 2 hours and overnight, and then concentrated in vacuo. The crude residue obtained was purified using flash chromatography on silica gel (DCM/methanol: 0 to 20%) to provide the expected product as a mixture of diastereoisomers. This mixture was purified using MS-preparative HPLC or by chiral HPLC to provide the separate purified diastereoisomers as solid compounds.

### (General Method B)

Step 1 - Preparation of Intermediate Compound C

To a solution of reagent B1 (1.2 eq) in DCM (25 mL/mmol) at room temperature was added a stirred suspension of compound A1 (1 eq) in a mixture of THF (12 mL/mmol) and acetonitrile (25 mL/mmol), followed by 1,8-diazabicyclo[5.4.0]undec-7-ene (2.38 eq.). The reaction mixture was allowed to stir at room temperature overnight. The reaction was monitored by LC/MS. The reaction mixture was concentrated in vacuo and purified using flash chromatography on silica gel (DCM/methanol: 0 to 20%) followed by preparative HPLC (H₂O/CH₃CN) to provide the expected compound Cl as mixture of diastereoisomers.

Step 2—Same method described in Step 2 of General Method A.

### (General Method C)

### Step 1

To a stirred solution of compound Al (1 eq.) in DCM (10 mL/mmol of Al) at 0° C. were added triethylamine (4 eq.) and reagent B1 (1.5 eq.). The reaction mixture was allowed to stir at 0° C. for 15 minutes and then was added N-methylimidazole (2 eq.). The reaction mixture was allowed to stir at room temperature overnight, then diluted with DCM, washed with water, brine and NH₄C1 solution. The organic layer was dried and concentrated in vacuo. The crude residue obtained was purified using flash chromatography on silica gel (DCM/methanol: 0 to 10%) to provide the expected intermediate.

### Step 2—Same as Step 2 of General Method A.

Compounds 1-5, depicted in Table 1 above were made using the above methods.

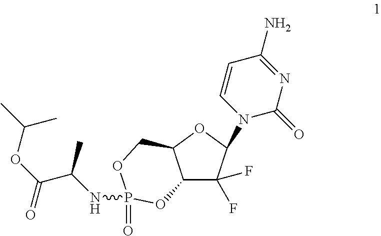

Compound 1 (Diastereoisomer 1): ¹NMR (DMSO-d₆, 400 MHz) δ (ppm) 7.56-7.48 (m, 3H), 6.36 (brs, 1H), 6.12-6.07 (m, 1H), 5.84 (d, J=7.05 Hz, 1H), 4.89 (heptuplet, J=6.26 Hz, 1H), 4.84 (brs, 1H), 4.66-4.58 (m, 1H), 4.55-4.48 (m, 1H), 4.27 (brs, 1H), 3.83-3.73 (m, 1H), 1.30 (d, J=7.24 Hz, 3H), 1.18 (d, J=6.22 Hz, 3H), 1.18 (d, J=6.22 Hz, 3H); ³¹P NMR (DMSO-d₆, 162 MHz) δ (ppm) 2.43 (s, 1P); ¹⁹F NMR (DMSO-d₆, 376 MHz) δ (ppm) −116.70 (brs, 2F); MS (ESI) m/z=439.0 (MH⁺).

Compound 1 (Diastereoisomer 2): ¹H NMR (DMSO-d₆, 400 MHz) δ (ppm) 7.83-7.82 (m, 1H), 7.45 (s, 2H), 6.37 (brs, 1H), 6.29 (dd, J=14.00 Hz, 9.98 Hz, 1H), 5.76 (d, J=7.34 Hz, 1H), 5.05 (brs, 1H), 4.88 (heptuplet, J=6.28 Hz, 1H), 4.62-4.49 (m, 2H), 4.14-4.09 (m, 1H), 3.79-3.69 (m, 1H), 1.25 (d, J=7.34 Hz, 3H), 1.20-1.17 (m, 6H); ³¹P NMR (DMSO-d₆, 162 MHz) δ (ppm) 5.11 (s, 1P); ¹⁹F NMR (DMSO-d₆, 376 MHz) δ (ppm) −116.64 (d, J=230 Hz, 1F), −117.40 (d, J=230 Hz, 1F); MS (ESI) m/z=439.0 (MH⁺).

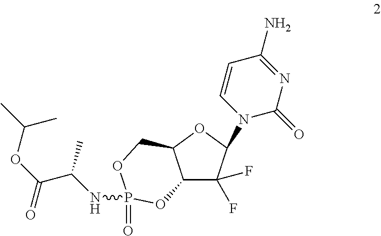

Compound 2 (Diastereoisomer 1): ¹H NMR (DMSO-d₆, 400 MHz) δ (ppm) 7.61 (d, J=7.54 Hz, 1H), 7.50 (brs, 2H), 6.39 (brs, 1H), 6.15-6.08 (m, 1H), 5.84 (d, J=7.47 Hz, 1H), 4.91 (heptuplet, J=6.23 Hz, 1H), 4.91-4.81 (m, 1H), 4.65-4.57 (m, 1H), 4.54-4.47 (m, 1H), 4.29 (brs, 1H), 3.83-3.73 (m, 1H), 1.29 (d, J=7.18 Hz, 3H), 1.20 (d, J=6.19 Hz, 3H), 1.19 (d, J=6.19 Hz, 3H); ³¹P NMR (DMSO-d₆, 162 MHz) δ (ppm) 2.68 (s, 1P); ¹⁹F NMR (DMSO-d₆, 376 MHz) δ (ppm) −116.12-(−117.18) (m, 2F); MS (ESI) m/z=439.0 (MH⁺).

Compound 2 (Diastereoisomer 2): ¹H NMR (DMSO-d₆, 400 MHz) δ (ppm) 7.83-7.82 (m, 1H), 7.46 (brs, 2H), 6.43-6.39 (m, 1H), 6.28 (dd, J=13.12 Hz and 9.95 Hz, 1H), 5.76 (d, J=7.51 Hz, 1H), 5.09-5.04 (m, 1H), 4.89 (heptuplet, J=6.34 Hz, 1H), 4.61-4.46 (m, 2H), 4.12 (brs, 1H), 3.80-3.69 (m, 1H), 1.25 (d, J=7.05 Hz, 3H), 1.195 (d, J=6.28 Hz, 3H), 1.19 (d, J=6.28 Hz, 3H); ³¹P NMR (DMSO-d₆, 162 MHz) δ (ppm) 5.32 (s, 1P); ¹⁹F NMR (DMSO-d₆, 376 MHz) δ (ppm) −116.19-(−117.58) (m, 2F); MS (ESI) m/z=439.0 (MH⁺).

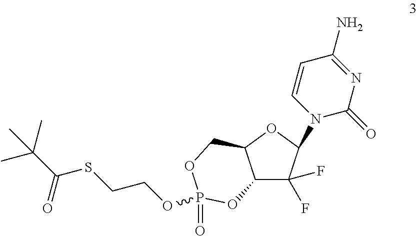

Compound 3 (Diastereoisomer 1): ¹H NMR (MeOD, 400 MHz) δ (ppm) 7.70 (d, J=7.51 Hz, 1H), 5.96 (d, J=7.63 Hz, 1H), 4.80-4.71 (m, 1H), 4.62 (t, J=9.99, 1H), 4.33-4.22 (m, 3H), 3.27 (t, J=6.16 Hz, 2H), 1.26 (s, 9H); ³¹P NMR (MeOD, 162 MHz) δ (ppm) −7.23 (s, 1P); MS (ESI) m/z=470.0 (MH⁺).

Compound 3 (Diastereoisomer 2): ¹H NMR (MeOD, 400 MHz) δ (ppm) 7.66 (d, J=7.68 Hz, 1H), 5.96 (d, J=7.68 Hz, 1H), 4.83-4.77 (m, 1H), 4.71-4.64 (m, 1H), 4.51-4.44 (m, 1H), 4.25 (t, J=6.29 Hz, 1H), 4.23 (t, J=6.29 Hz, 1H), 3.22 (t, J=6.41 Hz, 2H), 1.26 (s, 9H); ³¹P NMR (MeOD, 162 MHz) δ (ppm) −5.60 (s, 1P); MS (ESI) m/z=470.2 (MH⁺).

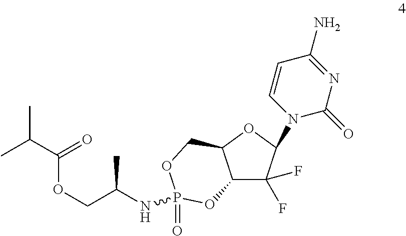

Compound 4 (Diastereoisomer 1): ¹H NMR (DMSO-d₆, 400 MHz) δ (ppm) 8.21 (brs, 1H), 8.03 (brs, 1H), 7.96-7.94 (m, 1H), 6.36 (brs, 1H), 5.89 (d, J=7.63 Hz, 1H), 5.89-5.83 (m, 1H), 5.08 (brs, 1H), 4.58-4.52 (m, 2H), 4.17-4.12 (m, 2H), 3.87-3.85 (m, 2H), 3.42-3.32 (m, 1H), 2.56-2.51 (m, 1H), 1.09 (d, J=6.94 Hz, 6H), 1.08-1.06 (m, 2H); ³¹P NMR (DMSO-d₆, 162 MHz) δ (ppm) 6.24 (s, 1P); ¹⁹F NMR (DMSO-d₆, 376 MHz) δ (ppm) −116.18-(−117.64) (m, 2F); MS (ESI) m/z=453.2 (MH⁺).

Compound 4 (Diastereoisomer 2): ¹H NMR (MeOD, 400 MHz) δ (ppm) 7.87-7.85 (m, 1H), 6.33 (brs, 1H), 6.12-6.09 (m, 1H), 5.03-4.96 (m, 1H), 4.75-4.68 (m, 1H), 4.57-4.50 (m, 1H), 4.45-4.39 (m, 1H), 4.07 (d, J=11.19 Hz and 5.22 Hz, 1H), 3.99 (d, J=11.11 Hz and 6.24 Hz, 1H), 3.64-3.48 (m, 1H), 2.62 (heptuplet, J=7.02 Hz, 1H), 1.25 (d, J=6.74 Hz, 3H), 1.18-1.17 (m, 6H); ³¹P NMR (MeOD, 162 MHz) δ (ppm) 5.09 (s, 1P); ¹⁹F NMR (MeOD, 376 MHz) δ (ppm) −118.64 (m, 2F); MS (ESI) m/z=453.0 (MH⁺).

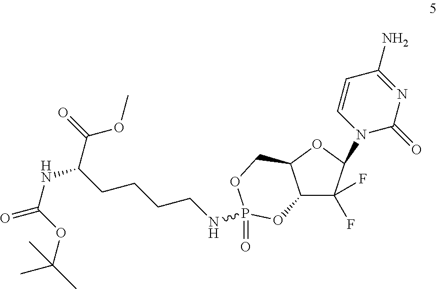

Compound 5 was synthesized according to General Method B. In this case, the diastereoisomers were isolated on step 1, and step 2 was carried out as follows: To a solution of pure diastereisomer of the appropriate compound C (0.66 mmol) in water (3.5 mL/mmol) was added formic acid (14 mL/mmol). The reaction mixture was allowed to stir at room temperature for 1 hour, and concentrated in vacuo. The crude residue obtained was purified successively by flash chromatography on silica gel (DCM/methanol: 0 to 10%), RP-18 chromatography (H₂O/CH₃CN), and MS-preparative HPLC (H₂O/CH₃CN) to provide each separate diasteromer of compound 5.

Compound 5 (Diastereoisomer 1): ¹H NMR (DMSO-d₆, 400 MHz) δ (ppm) 7.87-7.85 (m, 1H), 7.50-7.48 (m, 2H), 7.23 (d, J=7.66 Hz, 1H), 6.42-6.40 (m, 1H), 5.77 (d, J=7.56 Hz, 1H), 5.76-5.70 (m, 1H), 5.07-5.00 (m, 1H), 4.62-4.48 (m, 2H), 4.15-4.10 (m, 1H), 3.93-3.88 (m, 1H), 3.62 (s, 3H), 2.82-2.75 (m, 2H), 1.64-1.53 (m, 2H), 1.38 (s, 9H), 1.36-1.27 (m, 4H); ³¹P NMR (DMSO-d₆, 162 MHz) δ (ppm) 7.00 (s, 1P); ¹⁹F NMR (DMSO-d₆, 376 MHz) δ (ppm) −116.16-(−117.63) (m, 2F); MS (ESI) m/z=568.2 (MH⁺).

Compound 5 (Diastereoisomer 2): ¹H NMR (DMSO-d₆, 400 MHz) δ (ppm) 7.68 (d, J=7.59 Hz, 1H), 7.53-7.50 (m, 2H), 7.24 (d, J=7.63 Hz, 1H), 6.41 (brs, 1H), 5.83 (d, J=7.27 Hz, 1H), 5.56-5.53 (m, 1H), 4.81 (brs, 1H), 4.63-4.56 (m, 1H), 4.41 (brs, 1H), 4.30 (brs, 1H), 3.94-3.89 (m, 1H), 3.62 (s, 3H), 2.85-2.77 (m, 2H), 1.66-1.52 (m, 2H), 1.46-1.33 (m, 4H), 1.38 (s, 9H); ³¹P NMR (DMSO-d₆, 162 MHz) δ (ppm) 4.85 (s, 1P); ¹⁹F NMR (DMSO-d₆, 376 MHz) δ (ppm) −116.24 (s, 2F); MS (ESI) m/z=568.2 (MH⁺).

### Example 2

### Preparation of Compound 6

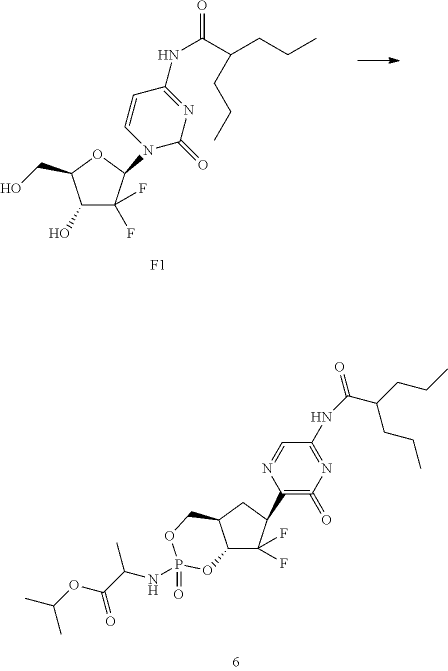

Compound 6 was synthesized from compound F1 and the appropriate compound of formula B1, using the methodology described in Example 1, General Method B.

Compound 6 (Diastereoisomer 1): ¹H NMR (DMSO-d₆, 400 MHz) δ (ppm) 11.15 (s, 1H), 8.33 (d, J=7.64 Hz, 1H), 7.33 (d, J=7.64 Hz, 1H), 6.42-6.34 (m, 2H), 5.13 (brs, 1H), 4.90 (heptuplet, J=6.20 Hz, 1H), 4.68 (brs, 1H), 4.64-4.55 (m, 1H), 4.25-4.20 (m, 1H), 3.82-3.71 (m, 1H), 2.68-2.60 (m, 1H), 1.58-1.48 (m, 2H), 1.39-1.31 (m, 2H), 1.28-1.19 (m, 13H), 0.88-0.84 (m, 6H); ³¹P NMR (DMSO-d₆, 162 MHz) δ (ppm) 5.22 (s, 1P); MS (ESI) m/z=565.2 (MH⁺).

Compound 6 (Diastereoisomer 2): ¹H NMR (DMSO-d₆, 400 MHz) δ (ppm)): ¹H NMR (DMSO-d₆, 400 MHz) δ (ppm) 11.18 (s, 1H), 8.13 (d, J=7.65 Hz, 1H), 7.40 (d, J=7.65 Hz, 1H), 6.40-6.34 (m, 1H), 6.20-6.14 (, 1H), 4.94-4.85 (m, 1H), 4.89 (heptuplet, J=6.20 Hz, 1H), 4.72-4.58 (m, 2H), 4.38 (brs, 1H), 3.84-3.74 (m, 1H), 2.68-2.61 (m, 1H), 1.58-1.49 (m, 2H), 1.39-1.31 (m, 5H), 1.28-1.18 (m, 10H), 0.88-0.84 (m, 6H); ³¹P NMR (DMSO-d₆, 162 MHz) δ (ppm) 2.53 (s, 1P); MS (ESI) m/z=565.2 (MH⁺).

### Example 3

### Preparation of Compound 7

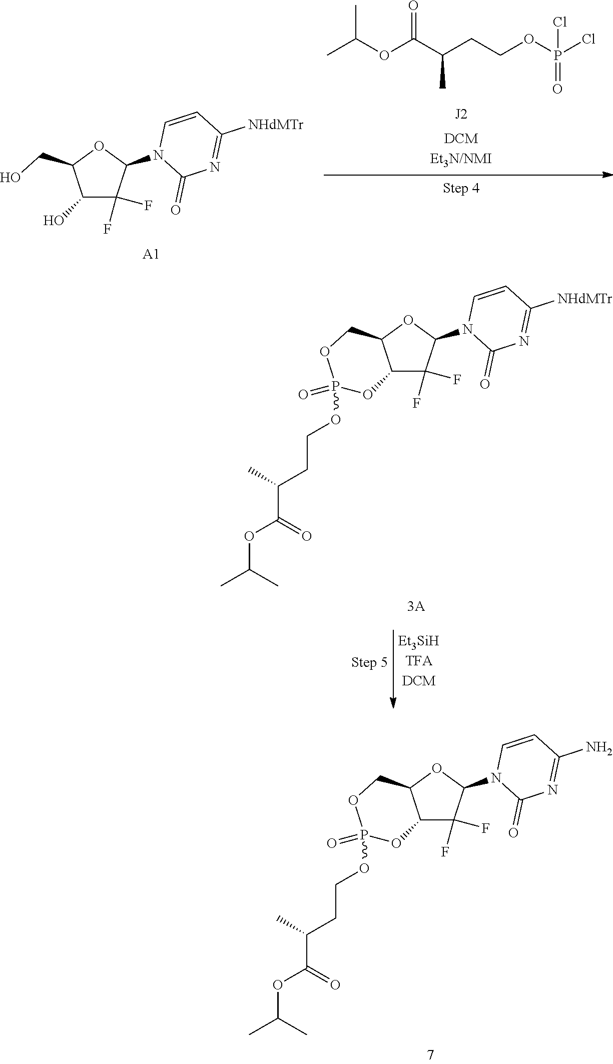

### Preparation of Intermediate J2

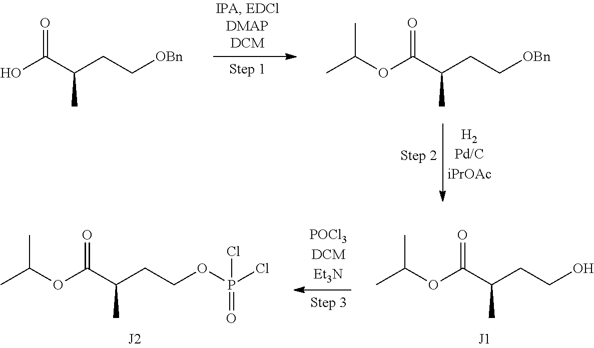 [SMILES: C[C@H](CCOCc1ccccc1)C(=O)O ; CC(C)OC(=O)[C@H](C)CCOCc1ccccc1 ; CC(C)OC(=O)[C@H](C)CCOP(=O)(Cl)Cl ; CC(C)OC(=O)[C@H](C)CCO]

### Step 1—Synthesis of isopropyl (R)-4-(benzyloxy)-2-methylbutanoate

To a stirred solution of (R)-4-(benzyloxy)-2-methylbutanoic acid (15.00 g, 72.00 mmol) in DCM (220 mL), was added isopropanol (55.2 mL, 0.72 mol) followed by the addition of a solution of N-(3-dimethylaminopropyl)-N′-ethylcarbodiimide hydrochloride (16.60 g, 86.44 mmol) in DCM (100 mL). The reaction mixture was allowed to stir, and then, 4-dimethylamino pyridine (0.88 g, 7.20 mmol) was added to the mixture. The reaction mixture was allowed to stir under nitrogen at room temperature overnight. The resulting reaction mixture was washed with water (250 mL). The organic layer was washed with a 10% solution of citric acid (×2) and with brine, then it was dried over sodium sulfate, filtered and concentrated in vacuo to provide isopropyl (R)-4-(benzyloxy)-2-methylbutanoate.

### Step 2—Synthesis of Intermediate J1

To a suspension of palladium on carbon (3.30 g, 31.00 mmol) in isopropyl acetate (50 mL) was added a solution of isopropyl (R)-4-(benzyloxy)-2-methylbutanoate (16.50 g, 66.00 mmol) in isopropyl acetate (500 mL). The reaction mixture was degassed few times with nitrogen. Then the flask was filled with hydrogen and it was allowed to stir under an atmosphere of hydrogen overnight. The reaction mixture was filtered through a pad of celite. The filtrates were concentrated in vacuo (water bath: 30° C.), and the resulting product was dried in vacuum oven for 4 h to provide the intermediate J1.

### Step 3—Synthesis of Intermediate J2

A solution of phosphorus (V) oxychloride (1.54 mL, 16.50 mmol) in anhydrous DCM (25 mL) was cooled to −10° C. A solution of intermediate J1 (2.40 g, 15.00 mmol) and triethylamine (2.1 mL, 15.00 mmol) in anhydrous DCM (25 mL) was added to the previous solution dropwise over a period of 2 h at −10/−5° C. The reaction mixture was allowed to stir at −10° C. overnight. The resulting reaction mixture was warmed to room temperature, and solvent was removed in vacuo. The residue obtained was dissolved in diethyl ether and filtered. The filtrate was concentrated in vacuo to provide intermediate J2, which was used in the next step without further purification.

### Step 4—Synthesis of intermediate 3A

To a solution of intermediate J2 in DCM (25 mL) was added dropwise over a period of 1.5 h a solution of 4-((bis(4-methoxyphenyl)(phenyl)methyl)amino)-1-((2R,4R,5R)-3,3-difluoro-4-hydroxy-5-(hydroxymethyl)tetrahydrofuran-2-yl)pyrimidin-2(1H)-one (6.00 g, 10.60 mmol) and triethylamine (5.1 mL, 36.50 mmol) in anhydrous DCM (25 mL). The reaction mixture was allowed to stir for 15 minutes, and 1-methylimidazole (5.72 mL, 71.70 mmol) was added. After 4 h stirring at room temperature, solvent was removed in vacuo and the resulting residue obtained was dissolved in ethyl acetate. The resulting solution was washed with water and with brine. The organic layer was dried over sodium sulfate, filtered and concentrated in vacuo. The crude residue obtained was purified using silica gel flash column chromatography (DCM/EtOAc: 20-40%) to provide intermediate compound 3A as a Sp/Rp mixture).

### Step 5—Synthesis of Compound 7

A solution of isopropyl (2R)-4-(((4aR,6R,7aR)-6-(4-((bis(4-methoxyphenyl)(phenyl)methyl)amino)-2-oxopyrimidin-1(2H)-yl)-7,7-difluoro-2-oxidotetrahydro-4H-furo[3,2-d][1,3,2]dioxaphosphinin-2-yl) oxy)-2-methylbutanoate (1.75 g, 2.27 mmol) and triethylsilane (2.5 mL, 16.13 mmol) in anhydrous DCM (35 mL) was cooled to 0° C. Trifluoroacetic acid (1.3 mL, 16.98 mmol) was added dropwise to the reaction mixture over a period of 25 min. After the addition, the reaction mixture was warmed to room temperature, stirred for 2 h, and then concentrated in vacuo (water bath: 30° C.). The crude residue obtained was azeotroped with toluene (×2), THF (×2), acetonitrile and chloroform, and then dried in the vacuum oven overnight. The residue obtained was purified using silica gel flash column chromatography (DCM/methanol 1-5%) to provide the 2 phosphorus isomers (Sp and Rp) of compound 7.

Compound 7 Diastereoisomer 1: ¹H NMR (DMSO-d₆, 400 MHz) δ (ppm) 7.82 (d, J=7.68 Hz, 1H), 7.77-7.71 (m, 2H), 6.44 (brs, 1H), 5.84 (d, J=7.68 Hz, 1H), 5.33 (brs, 1H), 4.89 (heptuplet, J=6.26 Hz, 1H), 4.77-4.70 (m, 1H), 4.65-4.63 (m, 1H), 4.40-4.38 (m, 1H), 4.17-4.09 (m, 2H), 2.00-1.92 (m, 1H), 1.77-1.69 (m, 1H), 1.19 (d, J=6.26 Hz, 3H), 1.185 (d, J=6.26 Hz, 3H), 1.10 (d, J=7.10 Hz, 3H); ³¹P NMR (DMSO-d₆, 162 MHz) δ (ppm) −5.74 (s, 1P); ¹⁹F NMR (DMSO-d₆, 376 MHz) δ (ppm) −116.75 (s, 2F); MS (ESI) m/z=468.4 (MIH⁺).

Compound 7 Diastereoisomer 2: ¹H NMR (DMSO-d₆, 400 MHz) δ (ppm) 8.05 (brs, 1H), 7.90 (brs, 1H), 7.85 (d, J=7.37 Hz, 1H), 6.44 (brs, 1H), 5.91 (d, J=7.60 Hz, 1H), 4.98 (brs, 1H), 4.89 (heptuplet, J=6.27 Hz, 1H), 4.77-4.69 (m, 1H), 4.56-4.52 (m, 1H), 4.33-4.28 (m, 1H), 4.19-4.11 (m, 2H), 2.62-2.53 (m, 1H), 2.08-1.99 (m, 1H), 1.84-1.76 (m, 1H), 1.185 (d, J=6.27 Hz, 3H), 1.18 (d, J=6.27 Hz, 3H), 1.12 (d, J=7.07 Hz, 3H); ³¹P NMR (DMSO-d₆, 162 MHz) δ (ppm) −7.48 (s, 1P); ¹⁹F NMR (DMSO-d₆, 376 MHz) δ (ppm) −115.48-(−117.01) (m, 2F); MS (ESI) m/z=468.2 (MH⁺).

### Example 4

### Preparation of Compound 8

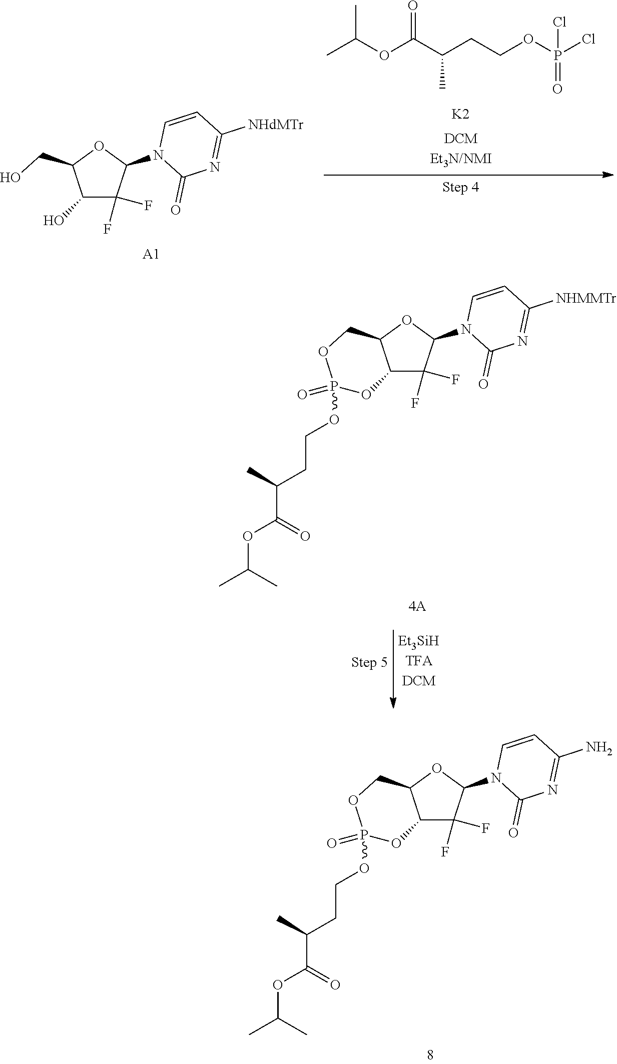

### Preparation of Intermediate K2

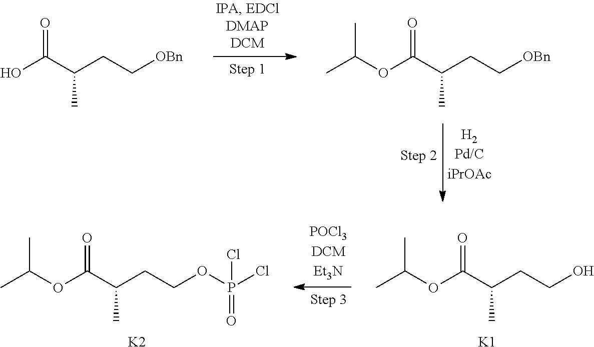

### Step 1—Synthesis of isopropyl (S)-4-(benzyloxy)-2-methylbutanoate

To a stirred solution of (S)-4-(benzyloxy)-2-methylbutanoic acid (30.00 g, 144.10 mmol) in DCM (400 mL), was added isopropanol (110.4 mL, 1.44 mol) followed by the addition of a solution of N-(3-dimethylaminopropyl)-N′-ethylcarbodiimide hydrochloride (33.20 g, 172.90 mmol) in DCM (100 mL). The reaction mixture was allowed to stir, and then, 4-dimethylamino pyridine (1.76 g, 14.40 mmol) was added to the mixture. The reaction mixture was allowed to stir under nitrogen at room temperature overnight. The resulting reaction mixture was washed with water (700 mL). The organic layer was washed with a 10% solution of citric acid (×2) and with brine, then it was dried over sodium sulfate, filtered and concentrated in vacuo to provide i sopropyl (S)-4-(benzyloxy)-2-methylbutanoate.

### Step 2—Synthesis of Intermediate K1

To a suspension of palladium on carbon (4.90 g, 46.04 mmol) in isopropyl acetate (700 mL) was added a solution of isopropyl (S)-4-(benzyloxy)-2-methylbutanoate (29.00 g, 115.84 mmol) in isopropyl acetate (200 mL). The reaction mixture was degassed few times with nitrogen. Then the flask was filled with hydrogen, and it was allowed to stir under an atmosphere of hydrogen for 2 h. The reaction mixture was filtered through a pad of celite. The filtrate wasconcentrated in vacuo (water bath: 30° C.), and the resulting product was dried in vacuum oven overnight to provide intermediate K1.

### Step 3—Synthesis of Intermediate K2

A solution of phosphorus (V) oxychloride (9.6 mL, 103.00 mmol) in anhydrous DCM (320 mL) was cooled to −10° C. A solution of intermediate K1 (15.00 g, 93.60 mmol) and triethylamine (13 mL, 93.60 mmol) in anhydrous DCM (160 mL) was added to the previous solution dropwise over a period of 4 h at −10/−5° C. The reaction mixture was allowed to stir at −10° C. overnight. The resulting reaction mixture was warmed to room temperature, and solvent was removed in vacuo. The residue obtained was dissolved in diethyl ether, filtered, and concentrated in vacuo to provide intermediate K2, which was used in the next step without further purification.

### Step 4—Synthesis of intermediate 4A

A solution of intermediate K2 in anhydrous DCM (150 mL) was cooled to 0° C. A solution of 1-((2R,4R,5R)-3,3-difluoro-4-hydroxy-5-(hydroxymethyl)tetrahydrofuran-2-yl)-4-(((4-methoxyphenyl)diphenylmethyl)amino)pyrimidin-2(1H)-one (38.50 g, 71.70 mmol) and triethylamine (32 mL, 0.23 mol) in anhydrous DCM (200 mL) was added dropwise over a period of 2 h to this solution, followed by the addition of 1-methylimidazole (5.72 mL, 71.70 mmol). The reaction mixture was warmed to room temperature and was followed by LCMS. After 4 h stirring at room temperature, solvent was removed in vacuo, and the resulting residue obtained was partitioned between ethyl acetate (1L) and water (500 mL). The aqueous layer was further extracted with ethyl acetate (500 mL). The combined organic layers were washed with water and brine, dried over sodium sulfate, filtered and concentrated in vacuo. The crude compound was purified using silica gel flash column chromatography (DCM/methanol 0-1%) to provide the intermediate compound 4A as a Sp/Rp mixture.

### Step 5—Synthesis of Compound 8

A solution of compound 4A (19.50 g, 26.40 mmol) and triethylsilane (27.9 mL, 0.18 mol) in anhydrous DCM (350 mL) was cooled to 0° C. Trifluoroacetic acid (24 mL, 313.40 mmol) was then added dropwise over a period of lh. The reaction mixture was warmed to room temperature, stirred for 3 h, and concentrated in vacuo (water bath: 30° C.). The residue obtained was azeotroped with toluene (×3), acetonitrile, THF and DCM, and then dried in the vacuum oven at 40° C. overnight. The crude compound was purified using silica gel flash column chromatography (DCM/methanol: 0-5%) to provide the 2 phosphorus isomers (Sp and Rp) of compound 8. A mixture of Sp/Rp isomers was further purified using 30% DCM in MeCN and then MeCN as a chromatography system to provide the individual Sp and Rp isomers of compound 8.

Compound 8 Diastereoisomer 1: The Diastereoisomer 1 was further purified using silica gel flash column chromatography (DCM/methanol: 0-5%), followed by prep-HPLC (Sunfire-Waters Prep C18 OBD 50×150, H₂O/CH₃CN (+0.02%HCOOH),over 60 min, flow 120 mL/min) to provide the expected compound. ¹H NMR (DMSO-d₆, 400 MHz) δ (ppm) 7.79 (d, J=7.38 Hz, 1H), 7.57-7.55 (m, 2H), 6.45 (brs, 1H), 5.80 (d, J=7.63 Hz, 1H), 5.32-5.29 (m, 1H), 4.89 (heptuplet, J=6.27 Hz, 1H), 4.76-4.69 (m, 1H), 4.65-4.63 (m, 1H), 4.39-4.38 (m, 1H), 4.17-4.09 (m, 2H), 2.01-1.92 (m, 1H), 1.77-1.69 (m, 1H), 1.19 (d, J=6.27 Hz, 3H), 1.18 (d, J=6.27 Hz, 3H), 1.10 (d, J=7.10 Hz, 3H); ³¹P NMR (DMSO-d₆, 162 MHz) δ (ppm) −5.75 (s, 1P); ¹⁹F NMR (DMSO-d₆, 376 MHz) δ (ppm) −116.72 (s, 2F); MS (ESI) m/z=468.5 (MH⁺).

Compound 8 Diastereoisomer 2: ¹H NMR (DMSO-d₆, 400 MHz) δ (ppm) 8.10 (brs, 1H), 7.90 (brs, 1H), 7.86 (d, J=7.65 Hz, 1H), 6.43 (brs, 1H), 5.91 (d, J=7.53 Hz, 1H), 4.98 (brs, 1H), 4.89 (heptuplet, J=6.29 Hz, 1H), 4.77-4.69 (m, 1H), 4.58-4.53 (m, 1H), 4.32-4.29 (m, 1H), 4.15 (dd, J=14.37 Hz, 6.80 Hz, 2H), 2.63-2.54 (m, 1H), 2.08-1.99 (m, 1H), 1.84-1.75 (m, 1H), 1.185 (d, J=6.29 Hz, 6H), 1.12 (d, J=7.05 Hz, 3H); ³¹P NMR (DMSO-d₆, 162 MHz) δ (ppm) −7.47 (s, 1P); ¹⁹F NMR (DMSO-d₆, 376 MHz) δ (ppm) −115.46-(−117.01) (m, 2F); MS (ESI) m/z=468.3 (MH⁺).

### Example 5

### Preparation of Compound 9

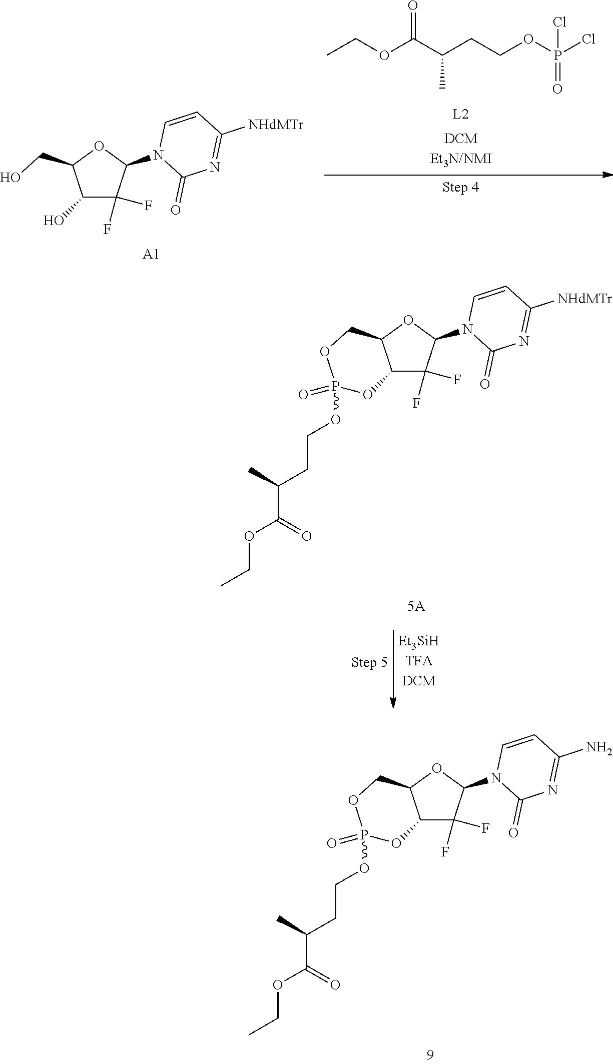

### Preparation of Intermediate L2

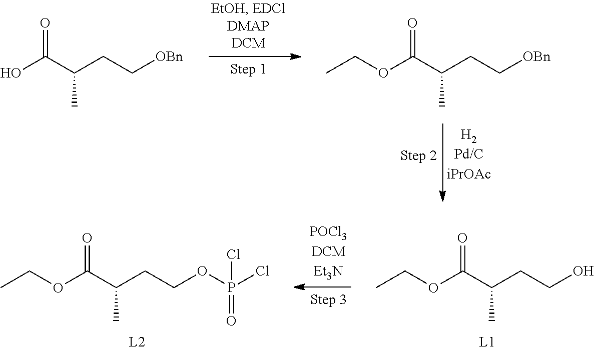

### Step 1—Synthesis of ethyl (S)-4-(benzyloxy)-2-methylbutanoate

To a solution of (S)-4-(benzyloxy)-2-methylbutanoic acid (14.00 g, 68.29 mmol) in DCM (200 mL) were added N-(3-Dimethylaminopropyl)-N′-ethylcarbodiimide hydrochloride (15.60 g, 81.94 mmol), 4-dimethylamino pyridine (0.83 g, 6.82 mmol) and ethanol (39.7 mL, 0.68 mol). The reaction mixture was allowed to stir under nitrogen at room temperature overnight. The resulting reaction mixture was washed with water (100 mL). The organic layer was washed with a 10% solution of citric acid (100 mL) and with a 1:1 mixture of brine and water (100 mL), then it was dried over sodium sulfate, filtered and concentrated in vacuo to provide ethyl (S)-4-(benzyloxy)-2-methylbutanoate.

### Step 2—Synthesis of intermediate L1

To a suspension of palladium on carbon (1.40 g, 13.15 mmol) in isopropyl acetate (350 mL) was added a solution of ethyl (S)-4-(benzyloxy)-2-methylbutanoate (14.2 g, 60.94 mmol) in isopropyl acetate (50 mL). The reaction mixture was degassed few times with nitrogen. Then the flask was filled with hydrogen and it was allowed to stir under an atmosphere of hydrogen for 7 h. The reaction mixture was filtered through a pad of celite. The filtrates were concentrated in vacuo to provide intermediate L1.

### Step 3—Synthesis of Intermediate L2

A solution of phosphorus (V) oxychloride (9.90 g, 64.71 mmol) in anhydrous DCM (70 mL) was cooled to −10° C. A solution of intermediate L1 (8.59 g, 58.83 mmol) and triethylamine (8.2 mL, 58.83 mmol) in anhydrous DCM (70 mL) was added to the previous solution dropwise at −10/−5° C. The reaction mixture was allowed to stir at −10° C. overnight. The reaction mixture was warmed to room temperature, and solvent was removed in vacuo. Diethyl ether was added to the residue, filtered and concentrated in vacuo to provide intermediate L2, which was used in the next step without further purification.

Step 4—Synthesis of intermediate 5A

To a solution of intermediate L2 in DCM (70 mL) was added dropwise a solution of 1-((2R,4R,5R)-3,3-difluoro-4-hydroxy-5-(hydroxymethyl)tetrahydrofuran-2-yl)-4-(((4-methoxyphenyl)diphenylmethyl)amino)pyrimidin-2(1H)-one (22.06 g, 41.18 mmol) and triethylamine (19.6 mL, 141.13 mmol) in anhydrous DCM (64 mL). The reaction mixture was allowed to stir for 30 min, and then, 1-methylimidazole (3.3 mL, 41.18 mmol) was added. The reaction mixture was allowed to stir at room temperature for 4 hours and concentrated in vacuo. The crude residue obtained was partitioned in ethyl acetate and water (350 mL). The aqueous layer was further extracted with ethyl acetate (×2). The combined organic layers were washed with brine, dried over sodium sulfate, filtered and concentrated in vacuo. The crude compound was purified using silica gel flash column chromatography (DCM/ethanol 2-3%) to provide the intermediate 5A as a Sp/Rp mixture.

### Step 5—Synthesis of compound 9

To a solution of ethyl (2S)-4-(((4aR,6R,7aR)-7,7-difluoro-6-(4-(((4-methoxyphenyl)diphenyl-methyl)amino)-2-oxopyrimidin-1(2H)-yl)-2-oxidotetrahydro-4H-furo[3,2-d][1,3,2]dioxaphosphinin-2-yl)oxy)-2-methylbutanoate (8.60 g, 11.86 mmol) in anhydrous DCM (250 mL) at 0° C. was added triethylsilane (9.5 mL, 61.03 mmol). A solution of trifluoroacetic acid (5.1 mL, 66.60 mmol) in DCM (20 mL) was added dropwise to the reaction mixture over a period of lh. The resulting reaction mixture was allowed to stir at 0° C. for 10 min and at room temperature for 2.5 h. Solvent was removed in vacuo and the resulting residue obtained was azeotroped with toluene (×2). The crude residue obtained was purified using silica gel flash column chromatography (DCM/ethanol 4-12%) to provide the Sp and Rp diastereomers of compound 9.

Compound 9 Diastereoisomer 1: ¹H NMR (DMSO-d₆, 400 MHz) δ (ppm) 7.98 (brs, 1H), 7.85-7.83 (m, 2H), 6.44 (brs, 1H), 5.89 (d, J=7.32 Hz, 1H), 4.98 (brs, 1H), 4.77-4.68 (m, 1H), 4.57-4.52 (m, 1H), 4.33-4.27 (m, 1H), 4.16 (dd, J=14.71 Hz, 6.74 Hz, 2H), 4.08 (q, J=7.05 Hz, 2H), 2.66-2.60 (m, 1H), 2.09-2.00 (m, 1H), 1.85-1.76 (m, 1H), 1.19 (t, J=7.05 Hz, 3H), 1.14 (d, J=7.05 Hz, 3H); ³¹P NMR (DMSO-d₆, 162 MHz) δ (ppm) −7.48 (s, 1P); ¹⁹F NMR (DMSO-d₆, 376 MHz) δ (ppm) −115.46-(−116.99) (m, 2F); MS (ESI) m/z=454.0 (MH⁺).

Compound 9 Diastereoisomer 2: The Diastereoisomer 2 was further purified using prep-HPLC (Sunfire-Waters Prep C18 OBD 50×150, H₂O/CH₃CN (+0.02%HCOOH),over 60 min, flow 120 mL/min) to provide the expected compound. ¹H NMR (DMSO-d₆, 400 MHz) δ (ppm) 7.78 (d, J=7.57 Hz, 1H), 7.54 (brs, 1H), 7.51 (brs, 1H), 6.46 (brs, 1H), 5.80 (d, J=7.59 Hz, 1H), 5.32-5.30 (m, 1H), 4.77-4.70 (m, 1H), 4.66-4.64 (m, 1H), 4.40-4.39 (m, 1H), 4.17-4.11 (m, 2H), 4.08 (q, J=7.07 Hz, 2H), 2.61-2.53 (m, 1H), 2.03-1.94 (m, 1H), 1.79-1.71 (m, 1H), 1.20 (t, J=7.07 Hz, 3H), 1.12 (d, J=7.07 Hz, 3H); ³¹P NMR (DMSO-d₆, 162 MHz) δ (ppm) −5.79 (s, 1P); ¹⁹F NMR (DMSO-d₆, 376 MHz) δ (ppm) −116.73 (s, 2F); MS (ESI) m/z=454.0 (MH⁺).

### Example 6

### Preparation of Compound 10

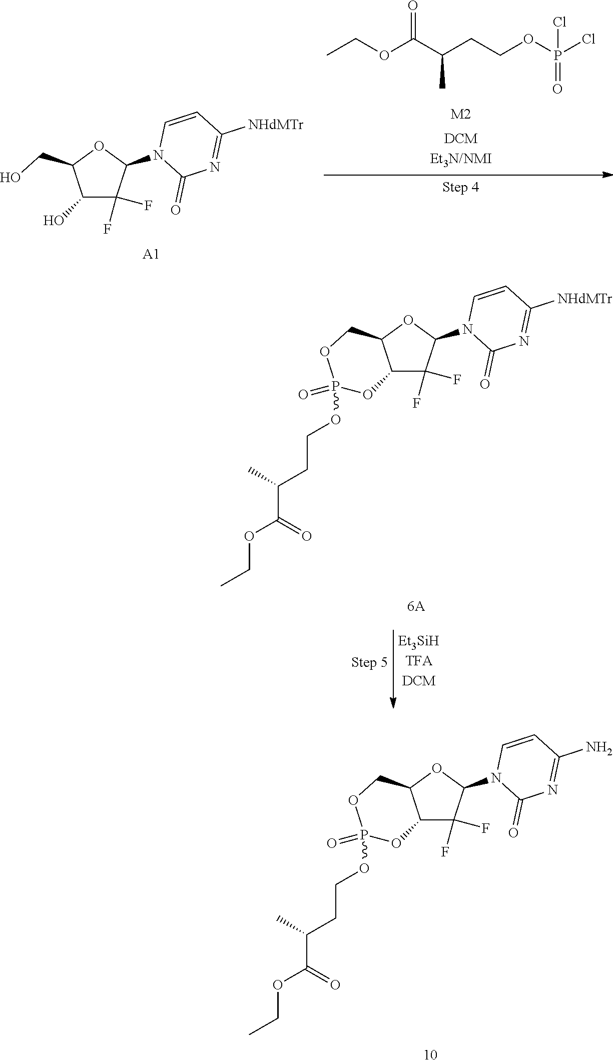

### Preparation of Intermediate M2

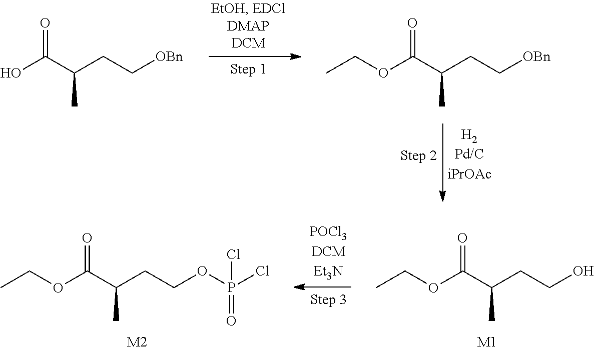

### Step 1—Synthesis of ethyl (R)-4-(benzyloxy)-2-methylbutanoate

To a solution of (R)-4-(benzyloxy)-2-methylbutanoic acid (11.43 g, 54.95 mmol) in DCM (170 mL) were added N-(3-Dimethylaminopropyl)-N′-ethylcarbodiimide hydrochloride (13.40 g, 70.16 mmol), 4-dimethylamino pyridine (0.69 g, 5.65 mmol) and ethanol (34 mL, 0.58 mol). The reaction mixture was allowed to stir under nitrogen at room temperature overnight.

The resulting reaction mixture was washed with water (100 mL). The organic layer was washed with a 10% solution of citric acid (100 mL) and with a 1:1 mixture of brine and water (100 mL), dried over sodium sulfate, filtered and concentrated in vacuo to provide ethyl (R)-4-(benzyloxy)-2-methylbutanoate.

### Step 2—Synthesis of intermediate M1

To a suspension of palladium on carbon (1.64 g, 15.41 mmol) in isopropyl acetate (400 mL) was added a solution of ethyl (R)-4-(benzyloxy)-2-methylbutanoate (15.34 g, 65.00 mmol) in isopropyl acetate (50 mL). The reaction mixture was degassed few times with nitrogen. Then the flask was filled with hydrogen and it was allowed to stir under an atmosphere of hydrogen for 7 h. The resulting reaction mixture was filtered through a pad of celite and washed with ethyl acetate (180 mL). The filtrates were concentrated in vacuo to provide intermediate M1.

### Step 3—Synthesis of intermediate M2

A solution of phosphorus (V) oxychloride (7.56 g, 64.04 mmol) in anhydrous DCM (68 mL) was cooled to -10° C. A solution of intermediate M1 (8.50 g, 58.22 mmol) and triethylamine (5.88 g, 58.22 mmol) in anhydrous DCM (34 mL) was added to the previous solution dropwise at −10/−5° C. The reaction mixture was allowed to stir at −10° C. overnight. The resulting reaction mixture was warmed to room temperature and solvent was removed in vacuo.

Diethyl ether was added to the residue, filtered and concentrated in vacuo to provide intermediate M2, which was used in the next step without further purification.

### Step 4—Synthesis of intermediate 6A

To a solution of 4-((bis(4-methoxyphenyl)(phenyl) methyl)amino)-1-((2R,4R,5R)-3,3-difluoro-4-hydroxy-5-(hydroxymethyl)tetrahydrofuran-2-yl)pyrimidin-2(1H)-one (20.00 g, 35.40 mmol) and triethylamine (14.11 g, 139.73 mmol) in anhydrous DCM (65 mL) was added a solution of intermediate L2 in DCM (80 mL). The reaction mixture was allowed to stir for 30 min at room temperature, and then, 1-methylimidazole (2.82 mL, 35.40 mmol) was added. After 4 h stirring at room temperature, the reaction mixture was concentrated in vacuo. The crude residue obtained was partitioned in ethyl acetate (200 mL) and water (200 mL). The aqueous layer was further extracted with ethyl acetate. The combined organic layers were dried over sodium sulfate, filtered and concentrated in vacuo. The crude compound was purified using silica gel flash column chromatography (DCM/EtOAc 40-50%) to provide compound 6A as a mixture of Sp and Rp isomers.

### Step 5—Synthesis of compound 10

To a solution of compound 6A (7.50 g, 9.92 mmol) in anhydrous dichloromethane (200 mL) at 0° C. was added triethylsilane (8.25 mL, 53.23 mmol). Trifluoroacetic acid (4.5 mL, 58.76 mmol) was added dropwise to the reaction mixture over a period of lh. The reaction mixture was allowed to stir at 0° C. for 10 min and then at room temperature for 1.5 h. Solvent was removed in vacuo and the resulting residue obtained was azeotroped with toluene (×2). The crude residue obtained was purified using silica gel flash column chromatography (DCM/EtOH 4-10%) to provide the Sp and Rp diastereomers of compound 10.

Compound 10 Diastereoisomer 1: ¹H NMR (DMSO-d₆, 400 MHz) δ (ppm) 8.04 (brs, 1H), 7.86-7.84 (m, 2H), 6.44 (brs, 1H), 5.90 (d, J=7.70 Hz, 1H), 5.02-4.92 (m, 1H), 4.78-4.69 (m, 1H), 4.56-4.52 (m, 1H), 4.34-4.27 (m, 1H), 4.20-4.13 (m, 2H), 4.07 (q, J=7.15 Hz, 2H), 2.65-2.60 (m, 1H), 2.09-2.00 (m, 1H), 1.85-1.77 (m, 1H), 1.18 (t, J=7.15 Hz, 3H), 1.14 (d, J=6.97 Hz, 3H); ³¹P NMR (DMSO-d₆, 162 MHz) δ (ppm) −7.50 (s, 1P); ¹⁹F NMR (DMSO-d₆, 376 MHz) δ (ppm) −115.49- (−117.02) (m, 2F); MS (ESI) m/z=454.0 (MH⁺).

Compound 10 Diastereoisomer 2: ¹H NMR (DMSO-d₆, 400 MHz) δ (ppm) 8.12 (brs, 1H), 7.94 (brs, 1H), 7.88 (d, J=7.81 Hz, 1H), 6.43 (brs, 1H), 5.89 (d, J=7.81 Hz, 1H), 5.36-5.33 (m, 1H), 4.78-4.71 (m, 1H), 4.66-4.61 (m, 1H), 4.44-4.37 (m, 1H), 4.18-4.11 (m, 2H), 4.07 (q, J=7.02 Hz, 2H), 2.59-2.53 (m, 1H), 2.02-1.93 (m, 1H), 1.79-1.70 (m, 1H), 1.19 (t, J=7.02 Hz, 3H), 1.12 (d, J=7.14 Hz, 3H); ³¹P NMR (DMSO-d₆, 162 MHz) δ (ppm) −5.70 (s, 1P); ¹⁹F NMR (DMSO-d₆, 376 MHz) δ (ppm) −116.13- (−117.35) (m, 2F); MS (ESI) m/z=454.0 (MH⁺).

### Example 7

### Preparation of Compound 11

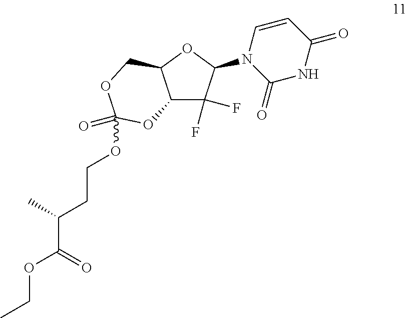

Compound 11 (diastereomer 1) was synthesized from 1-((2R,4R,5R)-3,3-difluoro-4-hydroxy-5-(hydroxymethyl)tetrahydrofuran-2-yl)pyrimidine-2,4(1H,3H)-dione and intermediate M2 according to the method described in scheme 6 above.

Compound 11 Diastereoisomer 1: ¹H NMR (DMSO-d₆, 400 MHz) δ (ppm) 11.73 (s, 1H), 7.87 (d, J=8.05 Hz, 1H), 6.39 (brs, 1H), 5.80 (d, J=8.05 Hz, 1H), 5.12-5.05 (m, 1H), 4.78-4.69 (m, 1H), 4.57-4.52 (m, 1H), 4.34-4.28 (m, 1H), 4.20-4.12 (m, 2H), 4.07 (q, J=7.10 Hz, 2H), 2.65-2.58 (m, 1H), 2.09-2.01 (m, 1H), 1.85-1.77 (m, 1H), 1.18 (t, J=7.10 Hz, 3H), 1.14 (d, J=7.10 Hz, 3H); ³¹P NMR (DMSO-d₆, 162MHz) δ (ppm) −7.54 (s, 1P); MS (ESI) m/z=453.2 (MH⁻).

### Example 8

### Preparation of Compound 12

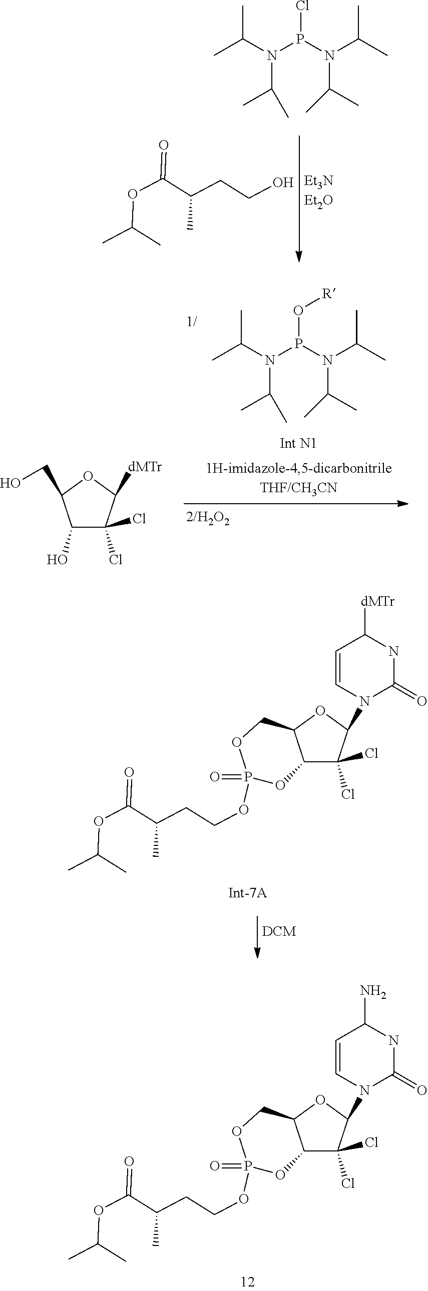

### Step 1—Synthesis of Intermediate N1

To a −15° C. solution of 1-chloro-N,N,N′,N′-tetraisopropylphosphinediamine (15 g, 56.2 mmol) in diethyl ether (187 mL), under nitrogen, was added triethylamine (7.84 mL, 56.2 mmol). A solution of isopropyl (S)-4-hydroxy-2-methylbutanoate (9.01 g, 56.2 mmol) in diethyl ether (94 mL) was added, and the reaction was allowed to stir at −15° C. for 1 hour and then at room temperature for 2 hours. The suspension was filtered under nitrogen and washed with diethyl ether. The filtrate was concentrated in vacuo at room temperature under nitrogen to provide intermediate compound N1, which was stored at -20° C. under nitrogen and was directly used in the next step without further purification: ³¹P NMR (162 MHz, CDCl₃) δ 123.9 (s, 1P).

### Step 2—Synthesis of Intermediate 7A

To a solution of THF (1.8 mL) under nitrogen at RT were simultaneously slowly added a solution of 2′-dichloro nucleoside (450 mg, 0.79 mmol) and 1H-imidazole-4,5-dicarbonitrile (234 mg, 1.98 mmol) (coevaporated 3 times with CH₃CN and THF) in THF (5.7 mL) and CH₃CN (2.9 mL), and a solution of the intermediate compound N1 (309 mg, 0.79 mmol) in THF (1.8 mL). The reaction mixture was stirred at RT overnight. Hydrogen peroxide (71 μL, 2.37 mmol) was then added dropwise at RT for 15 min. The reaction mixture was stirred at RT for 3 hours, and then, diluted with EtOAc and water. The aqueous layer was washed twice with EtOAc. The combined organic layers were washed with brine, dried, and concentrated under reduced pressure. The crude residue obtained was purified using flash chromatography on silica gel (DCM/MeOH: 0 to 10%) to provide compound 7A (MS (ESI) m/z=794.5 (MNa⁺).

### Step 3—Synthesis of Compound 12

Compound 12 was synthesized according to the method described in Example 5, Step 5 and using intermediate 7A as starting material. The resulting crude compound was purified using MS-preparative HPLC to afford the Sp and Rp diastereomers of compound 12. Compound 12 Diastereoisomer 1: ¹H NMR (DMSO-d₆, 400 MHz) δ (ppm) 7.80 (d, J=7.72 Hz, 1H), 7.54-7.50 (m, 2H), 6.72 (s, 1H), 5.84 (d, J=7.72 Hz, 1H), 4.91 (heptuplet, J=6.22 Hz, 1H), 4.86-4.83 (m, 1H), 4.76-4.64 (m, 2H), 4.25-4.14 (m, 3H), 2.65-2.56 (m, 1H), 2.10-2.01 (m, 1H), 1.86-1.78 (m, 1H), 1.195 (d, J=6.22 Hz, 3H), 1.19 (d, J=6.22 Hz, 3H), 1.13 (d, J=7.01 Hz, 3H); ³¹P NMR (DMSO-d₆, 162 MHz) δ (ppm) −7.26 (s, 1P); MS (ESI) m/z=500.2 (MH⁺).

### Example 9

### Replicon Activity and Cytotoxicity Assays

To measure cell-based anti-HCV activity of the compounds of the present invention, replicon cells (1b-Con1) are seeded at 5000 cells/well in 96-well plates one day prior to treatment with a compound of the invention. Various concentrations of a test compound of the invention in DMSO are then added to the replicon cells, with the final concentration of

DMSO at 0.5% and fetal bovine serum at 10% in the assay media. Cells are harvested three days post-dosing, and the replicon RNA level is determined using real-time RT-PCR (Taqman assay) with GAPDH RNA as endogenous control. EC₅₀ values are calculated from experiments with 10 serial twofold dilutions of the inhibitor in triplicate. To measure cytotoxicity in replicon cells of an inhibitor, an MTS assay is performed according to the manufacturer's protocol for CellTiter 96 Aqueous One Solution Cell Proliferation Assay (Promega, Cat # G3580) three days post dosing on cells treated identically as in replicon activity assays. CC₅₀ is the concentration of inhibitor that yields 50% inhibition compared to vehicle-treated cells. Cytotoxicity in other types of cells may be measured using the same MTS protocol.

Data were obtained using this method for selected compounds of the present invention, and are set forth below in Table 1. These data indicate that the compounds possess significant cytotoxicity windows over replicon activity.

### Example 10

### Determination of In Vivo Conversion of Prodrug to Nucleoside Triphosphate

The degree of conversion of a prodrug compound of the present invention to its corresponding nucleoside triphosphate (NTP) was measured in vivo using the procedure described below.

Liver samples were collected from either Wistar Hannover Rats or Beagle Dogs dosed with the prodrug via the freeze clamp procedure (animals anesthetized via isofluorane, the liver was clamped with modified clamps that are frozen in liquid nitrogen, then the clamped liver piece was placed in liquid nitrogen to ensure frozen completely; the liver clamp procedure was repeated to get a second piece of liver sample; samples stored at −80° C.). Liver samples were then homogenized using a a Spex Sample Prep Freezer/Mill (Cryomill); settings for the cryomill operation are 1 Cycle, 2 minute pre-chill time, 2 minute run time, 1 minute cool time, and a rate of 15 cycles/second (cps). Control liver samples collected from rats dosed with vehicle were cryomilled in the same manner. During this process it is imperative that anything that will come into contact with the liver samples remain frozen on dry ice at all times, such as all Cryomill sample containers/lids and spatulas.

The cryomilled control liver sample was used to generate the standard curve. An appropriate amount of cryomilled control liver sample was weighed out into a conical tube, depending on how many standard curves are needed, placed on wet ice and suspended with cold (approx. 0° C.) 70% Methanol / 30% (20mM EDTA/EGTA) that had been adjusted to pH 8 with sodium hydroxide at a ratio of 1:4 (liver:MeOH/EDTA-EGTA). The suspended liver homogenate was vortexed until a homogenous suspension was obtained. The standard curve ranged from 10 ng/mL to 50,000 ng/mL of NTP standard, as well as a QC sample at 10,000 ng/mL. A 500 μL aliquot of suspended control liver homogenate per each point on the standard curve and each QC was removed and placed into a 1.5 mL centrifuge tube, and 125 μL of each corresponding standard curve or QC standard solution was added to each individual control aliquot and re-vortexed. Liver sample aliquots were centrifuged at 4° C., 3645×g, for 10 minutes, and 450 μL of the supernatant was aliquoted into a 2 mL Square 96 well bioanalytical plate. Single and double blank samples are also generated from the suspended control liver homogenate using the procedure above, substituting the 125 μL of standard solution with 125 μL of water.

Approximately 1-2 grams of the cryomilled liver sample was weighed out into a 50 mL conical tube and placed on wet ice and suspended with cold 70% Methanol/30% (20mM EDTA/EGTA) that had been adjusted to pH 8 with sodium hydroxide at a ratio of 1:4 (liver:MeOH/EDTA-EGTA); the remaining cryomilled liver sample was stored at −80° C. for possible re-assay if needed. The suspended liver homogenate was vortexed until a homogenous suspension was obtained. A 500 μL aliquot of each unknown liver sample was removed and placed into a 1.5 mL centrifuge tube, and 125 μL of water was added to each aliquot and re-vortexed. Standard curve/QC liver sample aliquots were centrifuged at 4° C., 3645×g, for 10 minutes, and 450 μL of the supernatant was aliquoted into a 2 mL square 96 well bioanalytical plate, and an appropriate internal standard was added to all sample wells, standard curve/QC wells, and the single blank well. The sample plate was stored at −80° C. until analysis and results were reported in μM of NTP measured.

Results are provided in Table 2 below.

TABLE 1

| | | | NTP |
|---|---|---|---|
| | | | Mouse |
| | | CC50 | AUC0-24 |
| Compound | EC50 (1b) | (EDU) | 1mpk |
| No. | Range ⁽¹⁾ | Range ⁽²⁾ | Range ⁽³⁾ |
| 7 Diastereomer 1 | +++ | + | ND |
| 7 Diastereomer 2 | ++++ | + | ND |
| 8 Diastereomer 1 | +++ | + | ND |
| 8 Diastereomer 2 | +++ | + | ND |
| 9 Diastereomer 1 | +++ | + | ND |
| 9 Diastereomer 2 | +++ | + | ND |
| 10 Diastereomer 1 | +++ | + | ND |
| 10 Diastereomer 2 | +++ | + | ND |
| 11 Diastereomer 1 | + | + | ND |
| 11 Diastereomer 2 | + | + | nD |

⁽¹⁾ EC₅₀ is provided as follows: ++++ ≤ 250 nM, 250 nM < +++ ≤ 1 μM; 1 μM < ++ ≤ 10 μM; and + > 10 μM
⁽²⁾ CC₅₀ is provided as follows: + ≤ 50 μM and ++ > 50 μM
⁽³⁾ TP Mouse AUC0-24 1mpk is provided as follows: + ≤ 50; 50 < ++ ≤ 150; +++ > 150; and ND = Not Determined

### Uses of the Compounds of Formula(I)

### Treatment or Prevention of HCV Infection

The Compounds of Formula(I) are useful in the inhibition of HCV, the treatment of HCV infection and/or reduction of the likelihood or severity of symptoms of HCV infection and the inhibition of HCV viral replication and/or HCV viral production in a cell-based system. For example, the Compounds of Formula(I) are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one Compound of Formula(I) or a pharmaceutically acceptable salt thereof In a specific embodiment, the amount administered is effective to treat or prevent infection by HCV in the patient. In another specific embodiment, the amount administered is effective to inhibit HCV viral replication and/or viral production in the patient.

The Compounds of Formula(I) are also useful in the preparation and execution of screening assays for antiviral compounds. For example the Compounds of Formula(I) are useful for identifying resistant HCV replicon cell lines harboring mutations within NS5B, which are excellent screening tools for more powerful antiviral compounds. Furthermore, the Compounds of Formula(I) are useful in establishing or determining the binding site of other antivirals to the HCV NS5B polymerase.

The compositions and combinations of the present invention may be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b.

In one aspect, the present invention provides for the use of a compound of formula (I), or a pharmaceutically acceptable salt thereof, for inhibiting HCV NS5B activity or for preventing and/or treating infection by HCV in a patient in need thereof.

### Combination Therapy

In another embodiment, the present methods for treating or preventing HCV infection can further comprise the administration of one or more additional therapeutic agents which are not Cyclic Phosphate Substituted Nucleoside Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Cyclic Phosphate Substituted Nucleoside Derivative (which may include two or more different 2′-Substituted Nucleoside Derivatives), or a pharmaceutically acceptable salt thereof, and (ii) at least one additional therapeutic agent that is other than a Cyclic Phosphate Substituted Nucleoside Derivative, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Cyclic Phosphate Substituted Nucleoside Derivative and an additional therapeutic agent may be present in fixed amounts (dosage amounts) in a single dosage unit (e.g., a capsule, a tablet and the like).

In one embodiment, the at least one Cyclic Phosphate Substituted Nucleoside Derivative is administered during a time when the additional therapeutic agent(s) exert their prophylactic or therapeutic effect, or vice versa.

In another embodiment, the at least one Cyclic Phosphate Substituted Nucleoside Derivative and the additional therapeutic agent(s) are administered in doses commonly employed when such agents are used as monotherapy for treating a viral infection.

In another embodiment, the at least one Cyclic Phosphate Substituted Nucleoside Derivative and the additional therapeutic agent(s) are administered in doses lower than the doses commonly employed when such agents are used as monotherapy for treating a viral infection.

In still another embodiment, the at least one Cyclic Phosphate Substituted Nucleoside Derivative and the additional therapeutic agent(s) act synergistically and are administered in doses lower than the doses commonly employed when such agents are used as monotherapy for treating a viral infection.

In one embodiment, the at least one Cyclic Phosphate Substituted Nucleoside Derivative and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that may be treated or prevented using the combination therapy methods of the present invention include, but are not limited to, those listed above.

In one embodiment, the viral infection is HCV infection.

The at least one Cyclic Phosphate Substituted Nucleoside Derivative and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Cyclic Phosphate Substituted Nucleoside Derivative and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, one or more compounds of the invention are administered with one or more additional therapeutic agents, including but not limited to the therapeutic agents described, supra.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In another embodiment, one or more compounds of the present invention are administered with one additional therapeutic agent selected from an HCV protease inhibitor, an interferon, a pegylated interferon and ribavirin. In another embodiment, one or more compounds of the present invention are administered with one additional therapeutic agent selected from an HCV polymerase inhibitor, a viral protease inhibitor, an interferon, and a viral replication inhibitor. In another embodiment, one or more compounds of the present invention are administered with ribavirin, or a pharmaceutically acceptable salt thereof.

In still another embodiment, one or more compounds of the present invention are administered with two additional therapeutic agents selected from an HCV protease inhibitor, an HCV replication inhibitor, a nucleoside, an interferon, a pegylated interferon and ribavirin, or a pharmaceutically acceptable salt thereof.

In another embodiment, one or more compounds of the present invention are administered with an HCV protease inhibitor and ribavirin. In another specific embodiment, one or more compounds of the present invention are administered with a pegylated interferon and ribavirin, or a pharmaceutically acceptable salt thereof.

In another embodiment, one or more compounds of the present invention are administered with three additional therapeutic agents selected from an HCV protease inhibitor, an HCV replication inhibitor, a nucleoside, an interferon, a pegylated interferon and ribavirin, or a pharmaceutically acceptable salt thereof.

In one embodiment, one or more compounds of the present invention are administered with two additional therapeutic agents selected from an HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir, or a pharmaceutically acceptable salt thereof.

In a further embodiment, one or more compounds of the present invention are administered with ribavirin, interferon and an HCV polymerase inhibitor.

In another embodiment, one or more compounds of the present invention are administered with pegylated-interferon alpha and ribavirin, or a pharmaceutically acceptable salt thereof.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In one embodiment, the additional therapeutic agents comprise an HCV protease inhibitor and an HCV NS5A inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), RG7128 (Roche/Pharmasset), Sofosbuvir (Gilead), PSI-938 (Pharmasset-Gilead), PSI-879 (Pharmasset-Gilead), PSI-661 (Pharmasset), PF-868554/filibuvir (Pfizer), VCH-759/VX-759 (ViroChem Pharma/Vertex), HCV-371 (Wyeth/VirroPharma), HCV-796 (Wyeth/ViroPharma), IDX-184 (Idenix), IDX-375 (Idenix), NM-283 (Idenix/Novartis), MK-3682 (Merck), GL-60667 (Genelabs), JTK-109 (Japan Tobacco), PSI-6130 (Pharmasset), R1479 (Roche), R-1626 (Roche), R-7128 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3682 (Merck), VCH-222/VX-222 (ViroChem/Vertex), VCH-916 (ViroChem), VCH-716 (ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004), as well as pharmaceutically acceptable salts of any of the above agents.

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125; and the following compounds:

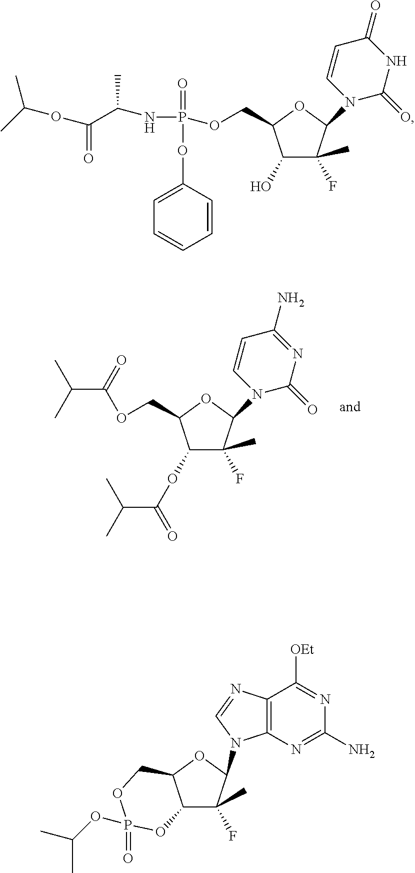

and pharmaceutically acceptable salts thereof.

Interferons useful in the present compositions and methods include, but are not limited to, interferon alfa-2a, interferon alfa-2b, interferon alfacon-1 and petroleum etherG-interferon alpha conjugates. “PEG-interferon alpha conjugates” are interferon alpha molecules covalently attached to a petroleum etherG molecule. Illustrative petroleum etherG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name petroleum etherG-Intron™ from Schering-Plough Corporation), interferon alpha-2b-XL (e.g., as sold under the trade name petroleum etherG-Intron™), interferon alpha-2c (Berofor Alpha™, Boehringer Ingelheim, Ingelheim, Germany), petroleum etherG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-a-2b-XL (Flamel Technologies), belerofon (Nautilus) and consensus interferon as defined by determination of a consensus sequence of naturally occurring interferon alphas (Infergen™, Amgen, Thousand Oaks, Calif.).

Examples of viral protease inhbitors useful in the present compositions and methods include, but are not limited to, an HCV protease inhibitor. Examples of HCV protease inhibitors useful in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), grazoprevir (Merck), BMS-650032 (Bristol-Myers Squibb), VX-985 (Vertex) and PHX1766 (Phenomix), as well as pharmaceutically acceptable salts of any of the above agents.

Viral replication inhibitors useful in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine, as well as pharmaceutically acceptable salts of any of the above agents.

HCV NS5A inhibitors useful in the present compositions and methods include, but are not limited to, ACH-2928 (Achilon), A-832 (Arrow Therpeutics), AZD-7295 (Astra Zeneca/Arrow), GS-5885 (Gilead), Ledipasvir (Gilead), Velpatasvir (Gilead), Samatasvir (Merck), PPI-461 (Presidio), PPI-1301 (Presidio), BMS-824383 (Bristol-Myers Squibb), BMS-790052 (Bristol-Myers Squibb), elbasvir (Merck) and ruzasvir (Merck), as well as pharmaceutically acceptable salts of any of the above agents. Additional HCV NS5A inhibitors useful as second additional therapeutic agents in the present compositions and methods include, but are not limited to those disclosed in International Publication No. WO 2010/111483.

HCV replicase inhibitors useful in the present compositions and methods include, but are not limited to, those disclosed in U.S. Patent Publication No. US20090081636.

The doses and dosage regimen of the other agents used in the combination therapies of the present invention for the treatment or prevention of HCV infection may be determined using the attending clinician, taking into consideration the approved doses and dosage regimen in the package insert; the age, sex and general health of the patient; and the type and severity of the viral infection or related disease or disorder. When administered in combination, the Cyclic Phosphate Substituted Nucleoside Derivative(s) and the other agent(s) may be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

Generally, a total daily dosage of the at least one Cyclic Phosphate Substituted Nucleoside Derivative(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

### Compositions and Administration

Due to their activity, the Compounds of Formula(I) are useful in veterinary and human medicine. As described above, the Compounds of Formula(I) are useful for treating or preventing HCV infection in a patient in need thereof.

When administered to a patient, the Compounds of Formula(I) may be administered as a component of a composition that comprises a pharmaceutically acceptable carrier or vehicle. The present invention provides pharmaceutical compositions comprising an effective amount of at least one Compound of Formula(I) and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules may be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Compounds of Formula(I) are administered orally.

In another embodiment, the one or more Compounds of Formula(I) are administered intravenously.

In one embodiment, a pharmaceutical preparation comprising a Compound of Formula(I) is in unit dosage form. In such form, the preparation is subdivided into unit doses containing effective amounts of the active components.

Compositions may be prepared according to conventional mixing, granulating or coating methods, respectively, and the present compositions can contain, in one embodiment, from about 0.1% to about 99% of the Compound of Formula(I)(s) by weight or volume. In various embodiments, the present compositions can contain, in one embodiment, from about 1% to about 70% or from about 5% to about 60% of the Compound of Formula(I)(s) by weight or volume.

The quantity of Compound of Formula(I) in a unit dose of preparation may be varied or adjusted from about 1 mg to about 2500 mg. In various embodiments, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Compounds of Formula(I) will be regulated according to the judgment of the attending clinician considering such factors as age, condition and size of the patient as well as severity of the symptoms being treated. Generally, a total daily dosage of the Compounds of Formula(I) range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Compound of Formula(I) or a pharmaceutically acceptable salt thereof; (ii) one or more additional therapeutic agents that are not a Compound of Formula(I); and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and two additional therapeutic agents, each of which are independently selected from the group consisting of HCV antiviral agents, immunomodulators, and anti-infective agents.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A method for treating a patient infected with HCV, said method comprising administering to said patient a compound of formula (I), or a pharmaceutically acceptable salt thereof, in an amount effective to treat infection by HCV in said patient, wherein formula (I) is: 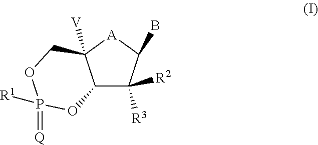
wherein:
A is O or S;
B is selected from: 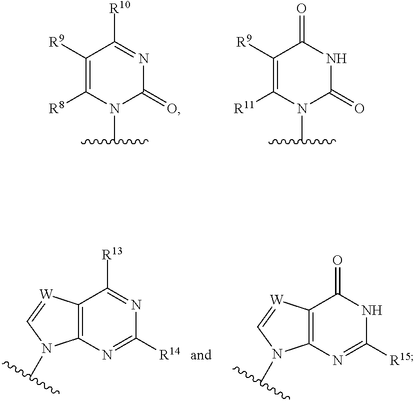
Q is O or S;
V is hydrogen, halogen or amino;
W is N, CH or CF;
R¹ is C₁-C₆ alkoxy, —O-(C₁-C₆ alkylene)-S—C(O)—(C₁-C₆ alkyl), 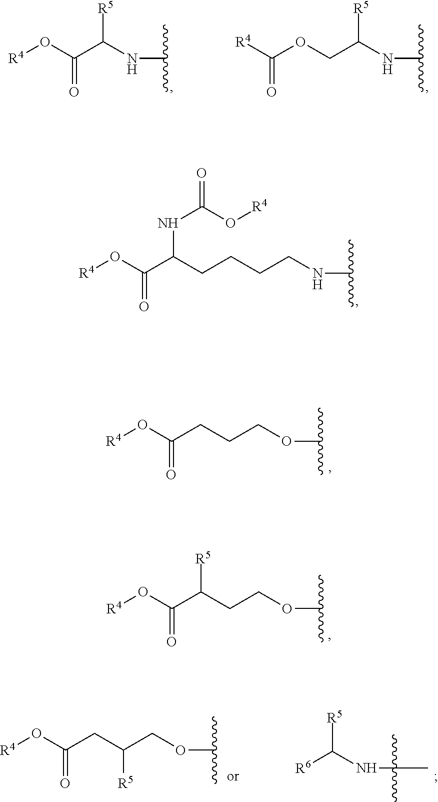
R² is halo;
R³ is halo;
each occurrence of R⁴ is independently selected from C₁-C₁₀ alkyl, C₃-C₁₀ cycloalkyl, aryl or —(C₁-C₆ alkylene)-aryl;
R⁵ is C₁-C₁₀ alkyl or —COOR⁷;
R⁶ is selected from —(C₁-C₁₀ alkylene)-C(O)O—(C₁-C₁₀ alkyl), —OC(O)—(C₃-C₁₀ cycloalkyl), aryl, aryloxy, —(C₁-C₁₀ alkylene)-aryl, 5 or 6-membered monocyclic heteroaryl , 9 or 10-membered bicyclic heteroaryl, —(C₁-C₁₀ alkylene)-(5 or 6-membered monocyclic heteroaryl) and —(C₁-C₁₀ alkylene)-(9 or 10-membered bicyclic heteroaryl);
R⁷ is C₁-C₁₀ alkyl, C₃-C₁₀ cycloalkyl, aryl or —(C₁-C₆ alkylene)-aryl;
R⁸, R⁹, R¹¹ and R¹² are each independently selected from H, C₁-C₆ alkyl, C₁-C₆ haloalkyl, C₁-C₆ hydroxyalkyl, halo, —OR¹⁶, —SR¹⁶ and —N(R¹⁶)₂ ;
R¹⁰, R¹³, R¹⁴ and R¹⁵ are each independently selected from H, C₁-C₆ alkyl, C₂-C₆ alkenyl, C₂-C₆ alkynyl, C₃-C₇ cycloalkyl, 5- or 6-membered monocyclic heteroaryl, 9- or 10-membered bicyclic heteroaryl, halo, —OR¹⁶, —SR¹⁶, —S(O)R¹⁶, —S(O)₂R¹⁶, —S(O)₂N(R¹⁶)₂, —NHC(O)OR¹⁶, —NHC(O)N(R¹⁶)₂, C₁-C₆ haloalkyl, C₁-C₆ hydroxyalkyl, —O—(C₁-C₆ haloalkyl), —CN, —NO₂, —N(R¹⁶)₂, —NH(C₁-C₆ alkylene)-(5- or 6-membered monocyclic heteroaryl), —NH(C₁-C₆ alkylene)-(9- or 10-membered bicyclic heteroaryl), —C(O)R¹⁶, —C(O)OR¹⁶, —C(O)N(R¹⁶)₂ and —NHC(O)R¹⁶, wherein said C₂-C₆ alkenyl group and said C₂-C₆ alkynyl group may be optionally substituted with halo;
each occurrence of e is independently selected from H, C₁-C₆ alkyl, C₁-C₆ haloalkyl, C₁-C₆ hydroxyalkyl, —(C₁-C₃ alkylene)ₘ- (C₃-C₇ cycloalkyl), —(C₁-C₃ alkylene)ₘ- (C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) and —(C₁-C₃ alkylene)ₘ-(9- or 10-membered bicyclic heteroaryl); and
each occurrence of m is independently 0 or 1.

2. The method of claim 1, wherein for the compound of formula (I), A is O.

3. The method of claim 1, wherein for the compound of formula (I), Q is O.

4. The method of claim 1, wherein for the compound of formula (I), R² is F.

5. The method of claim 1, wherein for the compound of formula (I), R³ is F.

6. The method of claim 1, wherein for the compound of formula (I), R² is Cl.

7. The method of claim 1, wherein for the compound of formula (I), R³ is Cl.

8. The method of claim 1, wherein for the compound of formula (I), B is selected from adenine, cytosine, guanosine or uracil.

9. The method of claim 1, wherein for the compound of formula (I), B is:

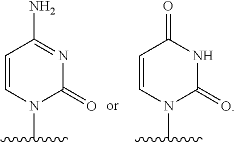 [SMILES: *n1ccc(N)nc1=O ; *n1ccc(=O)[nH]c1=O]

10. The method of claim 1, wherein for the compound of formula (I), V is H.

11. The method of claim 1, wherein for the compound of formula (I), V is F.

12. The method of claim 1, wherein the compound of formula (I) is selected from: 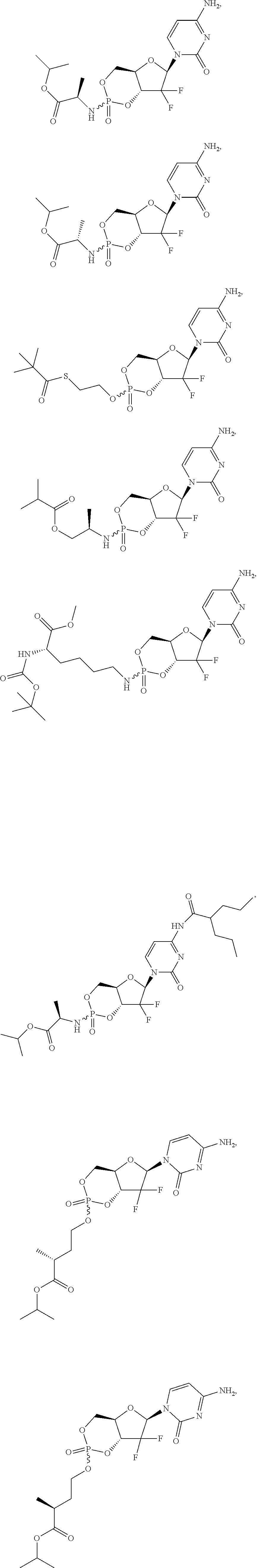 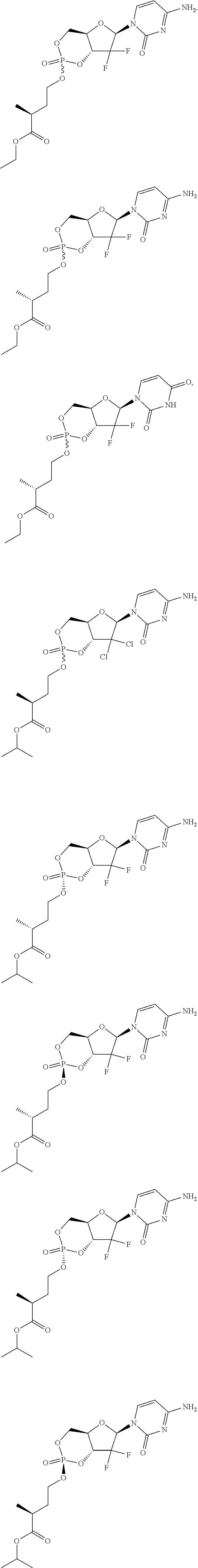 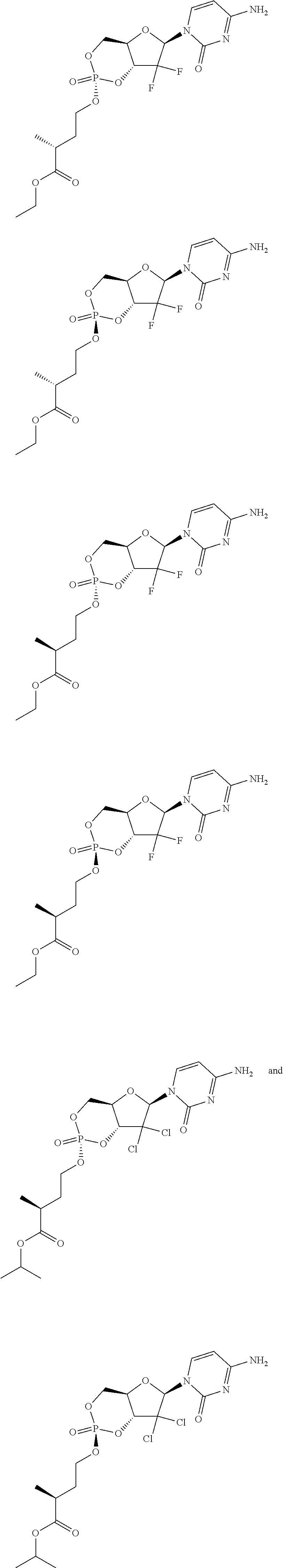
or a pharmaceutically acceptable salt thereof.

13. The method of claim 1, further comprising the step of administering to said patient a second therapeutic agent selected from the group consisting of HCV antiviral agents, immunomodulators, and anti-infective agents.

14. (canceled)

15. The use of a compound of formula (I) of claim 1, or a pharmaceutically acceptable salt thereof, for inhibiting HCV NS5B activity or for preventing and/or treating infection by HCV in a patient in need thereof.
